SEQUENCE LISTING (1 sequence)

SEQ ID NO: 1 - protein, 8 aa, Artificial Sequence
  Synthetic Polypeptide
DYKDDDDK

(19) United States
(12) Patent Application Publication
(10) Pub. No.: US 2019/0083522 A1
(43) Pub. Date: Mar. 21, 2019

(54) BIOSYNTHETIC MODULES

(71) Applicants: President and Fellows of Harvard College, Cambridge, MA (US); Dana-Farber Cancer Institute, Inc., Boston, MA (US)

(72) Inventors: William M. Shih, Cambridge, MA (US); Leo Chou, Brookline, MA (US); Rasmus Sorensen, Cambridge, MA (US); Jaeseung Hahn, Cambridge, MA (US)

(73) Assignees: President and Fellows of Harvard College, Cambridge, MA (US); Dana-Farber Cancer Institute, Inc., Boston, MA (US)

(21) Appl. No.: 16/083,712

(22) PCT Filed: Mar. 9, 2017
(86) PCT No.: PCT/US17/21546

§ 371 Date: Sep. 10, 2018

Related U.S. Application Data
(60) Provisional application No. 62/306,575, filed on Mar. 10, 2016.

(51) Int. Cl. A61K 31/711; A61K 31/7105; A61K 31/713; A61K 9/00; A61K 9/50; C12N 15/113; C12N 15/11; C12N 9/12

(52) U.S. Cl. CPC A61K 31/711; A61K 31/7105; A61K 31/713; A61K 9/0019; B82Y 5/00; C12N 15/113; C12N 15/111; C12N 9/1241; A61K 9/5068

(57) ABSTRACT

The present disclosure provides, in some aspects, nucleic acid-based biosynthetic modules for the production of ribonucleic acid (RNA) and other biopolymers.

### RELATED APPLICATION

This application claims the benefit under 35 U.S.C. § 119(e) of U.S. provisional application No. 62/306,575, filed Mar. 10, 2016, which is incorporated by reference herein in its entirety.

### FEDERALLY SPONSORED RESEARCH

This invention was made with government support under 1317694 and 1122374 awarded by National Science Foundation and under W911NF-12-1-0420 awarded by the U.S. Army. The government has certain rights in the invention.

### BACKGROUND

Ribonucleic acids (RNA) play a central role in biology by encoding for protein synthesis, catalyzing reactions, and regulating gene expression and activity. RNA-based therapeutics thus has the potential to treat a variety of diseases, including viral infections, cancers, and genetic disorders. Free RNA, however, has a short half-life in physiological fluids due to hydrolysis by nucleases and nonspecific binding to proteins and tissues.

### SUMMARY

Provided herein, in some aspects, are biosynthetic modules that enable efficient production of RNA in vivo, directly within a cell, by co-localizing the RNA synthesis machinery (e.g., deoxyribonucleic acid (DNA) template, RNA polymerase and, in some instances, associated enzymes (e.g., endonucleases/ribozymes)) within a synthetic (engineered) self-assembling protective nucleic acid (e.g., DNA) shell. In the presence of ribonucleotides (rNTPs), such as those present within the cell cytoplasm, biosynthetic modules are capable of transcribing RNA autonomously. Thus, biosynthetic modules may be used to deliver sufficient amounts of RNA (e.g., mRNA or siRNA) in vivo for therapeutic applications, for example. While the biosynthetic modules of the present disclosure are described primarily in the context of RNA synthesis, it should be understood that the modules may be used to synthesize other biopolymers, such as DNA and protein. In some embodiments, the biosynthetic modules are equipped with both transcription and translation machinery.

An example of a biosynthetic production module, as provided herein, is depicted in FIG. 1A. A nanocapsule comprising cylindrical barrels (e.g., 50-70 nm in diameter) stacked coaxially and capped at the two ends with hemispherical domes is assembled using, for example, a DNA origami approach (see, e.g., Rothemund, P. W. K. Nature 440 (7082): 297-302 (2006), incorporated by reference herein). Other nucleic acid nanostructure assembly methods (such as single-stranded tile (SST assembly)) may be used and are described elsewhere herein. The nucleic acid nanocapsule houses at specific locations RNA-synthesis units, each unit comprising an RNA polymerase and a circular DNA template. In the particular example of FIG. 1A, the nucleic acid nanocapsule also includes RNA processing enzymes (e.g., endoribonucleases) for maturation of the resultant RNA transcript (the DNA template used in this example is shown in FIG. 2A and is flanked by endonuclease cleavage sites). To enable co-localization of the polymerase and template, each molecule is coupled (linked) to an interior surface of the nanocapsule through short (e.g., less than 100 nucleotides in length) single-stranded nucleic acid tethers (oligonucleotides). One advantage of the biosynthetic modules is that the spatial configuration and copy number of the molecular components (e.g., polymerase, template and associated enzymes) can be precisely controlled to maximize RNA production yield.

To enable free rotation of the circular DNA template, which advantageously permits use of a rolling circle transcription (RCT) process, a novel DNA catenane (DNA leash) may be used. The DNA catenane, as provided herein, comprises circular DNA molecules that are sequence-independent relative to one another and are mechanically interlocked to one another, as depicted in FIG. 1A. This mechanical bond permits free rotation of a circular DNA template while co-localizing the template with the polymerase. Other molecular catenanes may also be used to permit free rotation of the template.

The biosynthetic modules of the present disclosure may be used, for example, to produce and deliver RNA interference molecules or mRNA inside cells, including at specific sub-cellular locations, such as the plasma membrane, cytoplasm or specific organelles; for cytoplasmic synthesis of self-regulatory RNA constructs and structures capable of responding to the presence of molecular species; as an in vitro or in vivo diagnostic device for the detection or identification of biomolecules; or as an in vitro cell-free expression system for production of RNA. Other applications and uses are encompassed by the present disclosure.

Thus, some aspects of the present disclosure provide an engineered nucleic acid nanostructure comprising a biopolymer synthesis unit that comprises a nucleic acid template encoding a product of interest and a polymerase co-localized to an interior surface of the nanostructure.

In some embodiments, the nanostructure is self-assembling. In some embodiments, the nanostructure is a deoxyribonucleic acid (DNA) nanostructure. In some embodiments, the nanostructure is in the shape of a nanocapsule. In some embodiments, the nanostructure has a spatial resolution of 20-500 nm.

In some embodiments, the polymerase is a ribonucleic acid (RNA) polymerase.

In some embodiments, the polymerase is linked to the interior surface of the nanostructure through chemical coupling using chemical groups. In some embodiments, the chemical groups are selected from amines, thiols, azides and alkynes. In some embodiments, the polymerase is linked to the interior surface of the nanostructure through single-stranded nucleic acid linkers. In some embodiments, the polymerase is linked to the interior surface of the nanostructure through biotin-streptavidin binding. In some embodiments, the polymerase is linked to the interior surface of the nanostructure through at least one genetically expressed tag. In some embodiments, the at least one genetically expressed tag is selected from SNAP-tags®, CLIP-tags™, ACP/MCP-tags, HaloTagd® and FLAG® tags.

In some embodiments, the nucleic acid template is a DNA template. In some embodiments, the nucleic acid template is circular. In some embodiments, the nucleic acid template is linked to the interior surface of the nanostructure. In some embodiments, the nucleic acid template is indirectly linked to the interior surface of the nanostructure through catenation with a nucleic acid catenane that is linked to the interior surface of the nanostructure. In some embodiments, the nucleic acid catenane is a DNA catenane comprising at least two sequence-independent circular DNA molecules.

In some embodiments, the nucleic acid template comprises a promoter operably linked to a nucleotide sequence encoding the product of interest. In some embodiments, the nucleotide sequence is flanked by ribonuclease recognition sites.

In some embodiments, the product of interest is an RNA. In some embodiments, the RNA is a messenger RNA (mRNA). In some embodiments, the RNA is an RNA interference molecule. In some embodiments, the RNA interference molecule is a short-hairpin RNA (shRNA) molecule or a short-interfering RNA (siRNA) molecule. In some embodiments, the RNA is a therapeutic RNA.

In some embodiments, the product of interest is a protein. In some embodiments, the protein is a therapeutic protein, a prophylactic protein or a diagnostic protein.

In some embodiments, the nanostructure further comprises at least one RNA processing molecule. In some embodiments, the at least one RNA processing molecule is linked to an interior surface of the nanostructure. In some embodiments, the at least one RNA processing molecule is selected from DNA endonucleases, RNA endoribonucleases, capping enzymes, ribosomes and ligases.

In some embodiments, the engineered nucleic acid nanostructure comprises at least two, at least five or at least ten RNA-synthesis units.

Also provided herein are cells comprising an engineered nucleic acid nano structure of the present disclosure (a biosynthetic module comprising at least one biopolymer synthesis unit).

In some embodiments, the nanostructure is located in cytoplasm of the cell. In some embodiments, the cell is a mammalian cell. In some embodiments, the mammalian cell is a human cell. In some embodiments, the mammalian cell is a cancerous cell.

Also provided herein, in some aspects, are methods of producing ribonucleic acid (RNA), comprising: introducing into a cell an engineered nucleic acid nano structure of the present disclosure; and incubating the cell under conditions that result in production of RNA.

In some embodiments, methods comprise introducing into a cell an engineered deoxyribonucleic acid (DNA) nanocapsule comprising a biopolymer synthesis unit that comprises (a) a RNA polymerase linked to an interior surface of the nanocapsule and (b) a circular DNA template comprising a promoter operably linked to a nucleotide sequence encoding a RNA of interest, wherein the circular DNA template is catenated with a nucleic acid catenane that is linked to the interior surface of the nanostructure; and incubating the cell under conditions that result in production of the RNA of interest.

In some embodiments, the nanocapsule further comprises at least one RNA processing molecule linked to an interior surface of the nanostructure. In some embodiments, the at least one RNA processing molecule is selected from DNA endonucleases, RNA endoribonucleases, capping enzymes, ribosomes and ligases.

Some aspects of the present disclosure provide methods of delivering a product of interest (e.g., a therapeutic ribonucleic acid) to a subject, comprising administering to a subject a nucleic acid nanostructure of the present disclosure, wherein the product of interest is a therapeutic molecule. In some embodiments, the therapeutic molecule is a therapeutic RNA. In some embodiments, the therapeutic RNA is a therapeutic messenger RNA (mRNA). In some embodiments, the therapeutic RNA is a therapeutic RNA interference (RNAi) molecule.

A further aspect of the present disclosure provides a biopolymer synthesis unit comprising a polymerase and a circular nucleic acid template, wherein the polymerase is tethered to a circular nucleic acid linker that is catenated to the circular nucleic acid template.

### BRIEF DESCRIPTION OF DRAWINGS

FIG. 1A shows a cross section of an example of a biosynthetic module comprising RNA-synthesis units. Each cylinder represents a nucleic acid duplex, and each ribbon represents a single-stranded nucleic acid. An RNA-synthesis unit, in this example, comprises a tethered RNA polymerase (for example, T7 RNAP) and a circular DNA template catenated to a DNA catenane, which itself is tethered to an interior surface of the a nucleic acid nanocapsule. RNA-processing enzymes (for example, Cas6e) are tethered towards the oculi of the nanocapsule for RNA modification before RNA transcripts exit the nanocapsule. FIG. 1B is a transmission electron microscopy (TEM) of a DNA nanocapsule and fluorescence micrograph of DNA barrels taken up by SKOV-3 cells.

FIG. 2A shows an example of a DNA template encoding a short hairpin RNA (shRNA) of interest flanked by 5′ Cas6e and 3′ HDV cleavage sites. FIG. 2B shows RNA multimers produced using rolling circle transcription (RCT) and processed into functionally active hairpin monomers.

FIG. 3 shows shRNA-knockdown of luciferase activity in cancer cells.

FIG. 4A shows an example of a method for producing nucleic acid catenanes using sequence-independent DNA circles. Each line represents single-stranded DNA, and black dots between lines indicate duplex formation. Linear DNA strands intended for circularization (longer, darker gray strands) are folded into a nanostructure with desired topology by hybridizing to a linear DNA scaffold or “brace strands” (shorter, e.g., less than 100 nt, lighter gray strands). Following ligation, which is used to form the mechanical bond, the structure is denatured, and interlocked DNA circles are purified. FIG. 4B shows the gels from the resulting purification products.

FIG. 5 shows an illustration of a long single-stranded DNA scaffold-mediated control of target DNA topology generated using caDNAno software. The two catenating DNA strands (light gray and medium gray) are positioned next to each other along the helical direction using a single, scaffolding DNA strand. Additional brace strands can be added to increase folding efficiency and product yield.

FIGS. 6A-6D show an example of short single-stranded DNA braces-mediated control of target DNA topology. FIG. 6A shows an illustration of DNA nanostructure architecture generated using caDNAno software (one long strand is a 84 nt strand, and the other long strand is a 86 nt strand; the shorter strands are “braces”). FIG. 6B shows a denaturing PAGE after DNA folding and ligation to form a catenane. FIG. 6C shows a denaturing PAGE purified of catenane following digestion using a restriction enzyme. The 86-nt strand has an EcoRI site, and the 84-nt strand has a HindIII site. Digestion of the catenane with one enzyme produced a digested linear strand and an undigested circular strand, and double digestion resulted in two linear strands. (L=86 nt strand ligated with circularizing splint, T=84 nt strand ligated with circularizing splint, NS=ligated nanostructure, C=purified catenane, E13=digested catenane with indicated restriction enzyme). FIG. 6D shows an interlocked circular DNA template (light grey) with a DNA leash (dark grey) in the form of a catenane, which can then be tethered to solid supports while enabling the template to rotate freely for rolling-circle transcription.

FIG. 7 depicts an integrated RNA nanocapsule (an example of a biosynthetic module). A 120 nm by 30 nm DNA nanostructure housing the RNA polymerases (light grey structures), DNA templates (large grey circles), and RNA processing enzymes (dark grey structures). When the structure enters a cell with the appropriate starting materials, therapeutic RNA molecules are produced. The DNA templates should be free to rotate in order to maximize RNA synthesis rate, but must still be tethered to the structure so they do not leak out. This is accomplished by attaching the templates via an interlocked ring (dark grey circles), which is integrated into the main structure. After transcription, the RNA is processed by enzymes attached next to the RNA polymerase to produce a therapeutic RNA product.

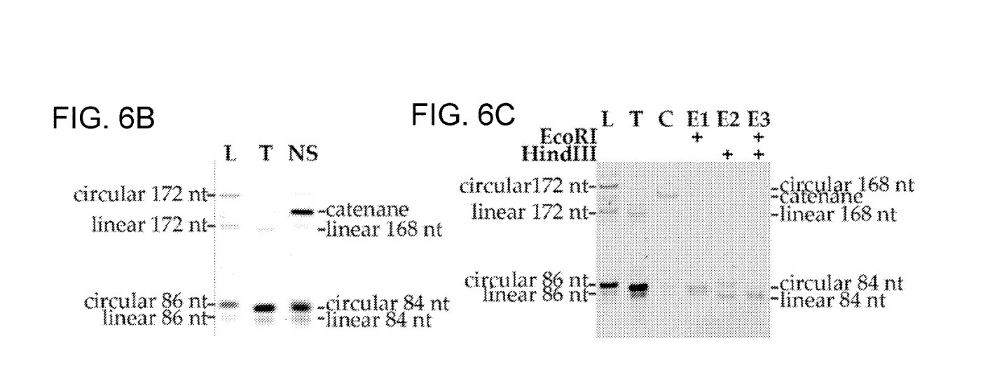

FIGS. 8A-8B show single-guide RNA (sgRNA)-mediated gene silencing. FIG. 8A shows rolling-circle transcription, which generates multimeric transcripts (e.g., 1X, 2X, 3X, . . . etc.) in the absence of RNA processing. Addition of Cas6e generates the functional shRNA. FIG. 8B shows luciferase knockdowns in SKOV-3 cells. Three shRNAs targeting the luciferase gene were generated by in vitro transcription and Cas6e processing. They silenced the luciferase gene as effectively as the positive (+′ve) siRNA control.

FIG. 9 shows a DNA origami nanocapsule schematic (right) and TEM images of the structure (left).

FIG. 10 shows the DNA design of a nanocapsule (top, left) and a TEM image (top, right) showing the structure's potential use as a “protein vault.” The structure's integrity is pH-dependent, as shown by the three lower images.

FIG. 11 shows that the combination of RNA transcription and processing (“circular+cleavage”) maximizes yield.

FIG. 12 is a schematic depicting spatial organization of RNA-extrusion machinery in one embodiment.

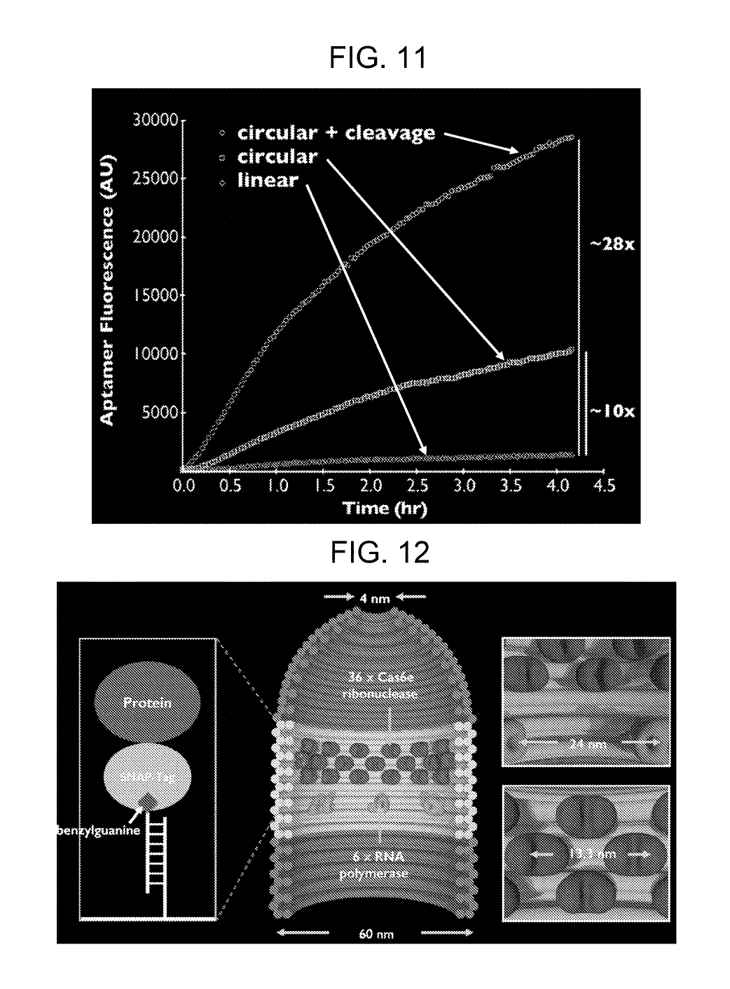

FIG. 13 illustrates a DNA barrel with six handles for RNA polymerase. In this two-plex embodiment, the handle (binding site) is dark grey and the polymerase (T7 RNA polymerase (RNAP)) is light grey. Six binding sites and T7 can be resolved.

FIG. 14A shows tethered RNA polymerase transcription using a nanocapsule system. A schematic of the DNA origami used is presented in the lower left corner. The graph shows that the combination of DNA barrel and tethered RNA polymerase yields the most RNA polymerase activity. FIG. 14B shows the efficiency of RNA production between the nanocapsule system, in vitro transcription in solution (denoted as “+”), and DNA origami without binding sites (denoted as “−”). The left graph shows RNA production (as measured by normalized fluorescence) and the right graph depicts the rate of RNA production.

FIG. 15 shows integrated RNA manufacturing, including programmed transcription and processing using handle designs.

FIG. 16 depicts an example of template integration and catenane production.

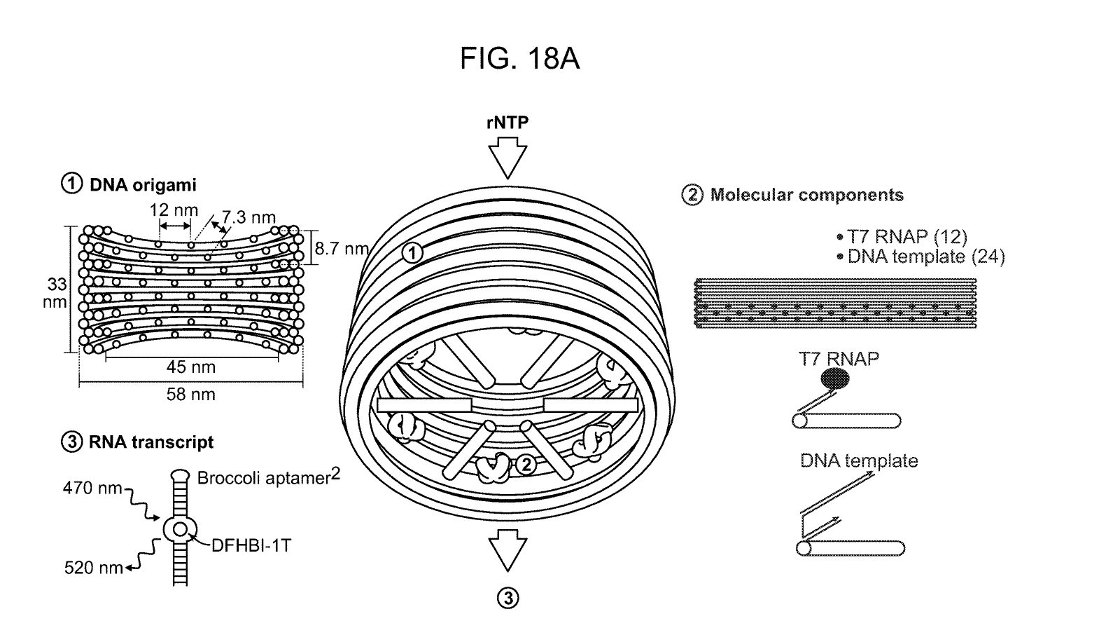

FIG. 17 shows intracellular delivery using a polymer coating.

FIG. 18A is a schematic illustrating a nanocapsule system using RNA polymerase and DNA templates. FIG. 18B is a schematic illustrating the synthesis and analysis of the biosynthetic nanocapsule.

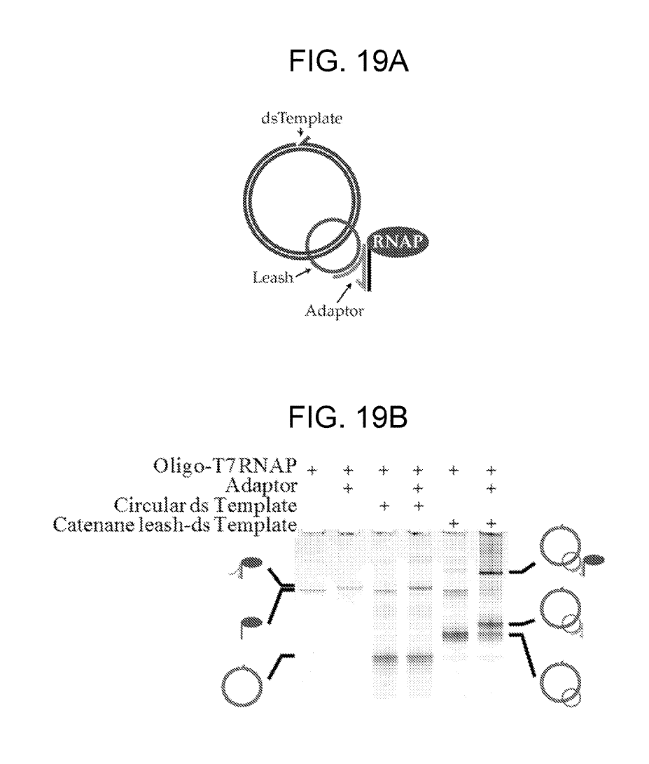

FIGS. 19A-19C show another example of a system for tethered transcription. FIG. 19A is an illustration of the components of the system. A dsDNA template is catenated with a leash strand, and the adaptor strand hybridizes to both the leash strand and an oligo-conjugated T7 RNA polymerase (RNAP). FIG. 19B shows a photograph of an SDS-PAGE verifying construction of the system. FIG. 19C shows results representative of system performance.

### DESCRIPTION

Provided herein, in some aspects, are biosynthetic modules and methods that enable efficient in vivo production of biopolymers, such as DNA, RNA and protein. Components of biosynthetic modules include, for example, biopolymer synthesis units tethered to the interior surface of a nucleic acid nanostructure, such as a nucleic acid nanocapsule. While the nucleic acid nanostructures of the present disclosure are described primarily in the context of nucleic acid nanocapsules, it should be understood that the biosynthetic modules are not so limited. Modules may include any nucleic acid nanostructure that forms an exterior shell and an interior void compartment to which biopolymer synthesis units may be tethered.

### Exemplary Gene-Regulating RNA-Producing Nanocapsule

The nanocapsules of the present disclosure, in some embodiments, enable the prolonged or shorter periods of RNA (e.g., mRNA or RNAi) expression (e.g., in vivo) relative to conventional RNA-delivery therapies. Rather than injecting RNA into the blood, these provided herein are small nanocapsules capable of synthesizing RNA the capsule enters a cell. Advantageously, the nanocapsules can be programmed to enter only a particular type of cell. Further, transcription from nanocapsule can me modulated, as needed (e.g., to terminate transcription at a particular time or in a particular environment (e.g., in response to a change in pH or other intracellular signal).

Nanocapsules are produced, in some embodiments, using DNA as the main structural building block. DNA strands of the correct sequence are synthesized and mixed at the correct temperature (e.g., room temperature), for example. The DNA then assembles itself into structures of almost any desired shape, with sizes similar to that of a virus, in some instances. Using DNA as a building material has several advantages over traditional nano-engineering materials such as polymers, lipids, or proteins: producing a new structure of a certain shape is typically much faster with DNA. Other components, such as proteins or polymers, can be tethered to the DNA structure, either inside or outside the structure, in a precise, controlled manner.

One non-limiting example is a 100x60 nanometer capsule-shaped nanostructure, formed by stacked DNA rings with half-spheres at the ends. This “DNA capsule” (DNA nanocapsule) has a spacious cavity where components can be organized while enjoying some level of protection from the exterior environment. The structure may be stabilized using oligo-lysine, a small polypeptide which protects the DNA structure from DNases and renders it stable in the physiological environment encountered in the blood and inside cells. Lipids, proteins or other components can be added to the outside the structure, in some embodiments, which protect the structure as it travels to an intended location.

One challenge of using large, complex structures, such as capsules, is that the number of structures per cell is much lower than the number of molecules typically required for traditional medicinal drugs. Even if a capsule were to be filled completely, the therapeutic effect of a single capsule would be limited. Instead of simply stuffing capsules with drugs, for example, the nanocapsules of the present disclosure, in some embodiments, include enzymes attached to the inside of the capsule. Each of these enzymes can then synthesize a high number of therapeutic molecules, producing a much larger amount than could be packed into a single capsule. This process often uses multiple enzymes and other components to work together to produce the desired product. The DNA nanocapsules as provided herein efficiently facilitate organization of the required components.

RNA is one example of a therapeutic molecule of particular interest. RNA is a natural and important part of every cell. The most important function of RNA is to carry the genetic information from the chromosomal DNA inside the nucleus of the cell to the main cellular environment, where the RNA molecules are translated to proteins. Proteins, in turn, are responsible for carrying out the majority of actual function inside and outside the cell. The RNA at the level between the DNA-encoded genetic information and the functional proteins serves as more than just a messenger: a range of RNA-based systems is responsible for regulating and converting the genomic messages. One example of a RNA-regulatory system is RNA interference. In RNA interference, a short RNA duplex provides instructions guiding an RNA-degradation complex, which can selectively degrade target messenger RNA.

Nanocapsules of the present disclosure, in some embodiments, have been equipped with machinery produce such short RNA hairpin duplexes. The machinery includes, for example, a DNA template encoding a RNA sequence, a RNA polymerase, which produces a single, long strand of RNA, and an RNA endonuclease, which cuts the long RNA string into multiple small RNA hairpin duplexes. Once the nanocapsule is inside a cell, the machinery begins to synthesize the RNA duplexes, which can then guide the degradation of other RNA molecules.

The RNA hairpin duplexes produced by the nanocapsule can be used to regulate the expression of any gene of interest, for example. This is an attractive approach for transiently evaluating the effect of a particular gene-therapy target. For instance, many cancer cells produce different proteins, which help the cancer cells survive, proliferate and become malignant. The nanocapsule can also produce messenger RNA, which will increase the amount of a given protein. Therefore, it is possible to both increase and decrease the level of any given protein.

RNA products can also be used for other applications. Immunotherapy uses the body's own immune system to treat diseases, and works by teaching immune cells to respond to particular targets. Immunotherapy is believed to have great potential for cancer treatment, where immune cells can be taught to respond to cancer cells. A critical part of the immune cell-training program is conditional stimulation, where some effective stimulant is used to create a connection with the target of choice. Double-stranded RNA is a very potent immune cell simulant, and the RNA nanocapsule may be used to activate immune cells for use in immunotherapy.

### Biopolymer Synthesis Units

Biopolymer synthesis units include molecular components (e.g., templates, enzymes, tethers, etc.) for producing (synthesizing) a biopolymer of interest. In some embodiments, a biopolymer synthesis unit comprises a nucleic acid template encoding a product of interest. For example, a biopolymer synthesis unit, in one embodiment, is an RNA synthesis unit—that is, the unit transcribes RNA from a DNA template. A RNA synthesis unit may include, for example, a DNA template and polymerase, each tethered (directly or indirectly) to an interior surface of a nucleic acid nanocapsule (or other nucleic acid nanostructure).

### Nucleic Acid Template

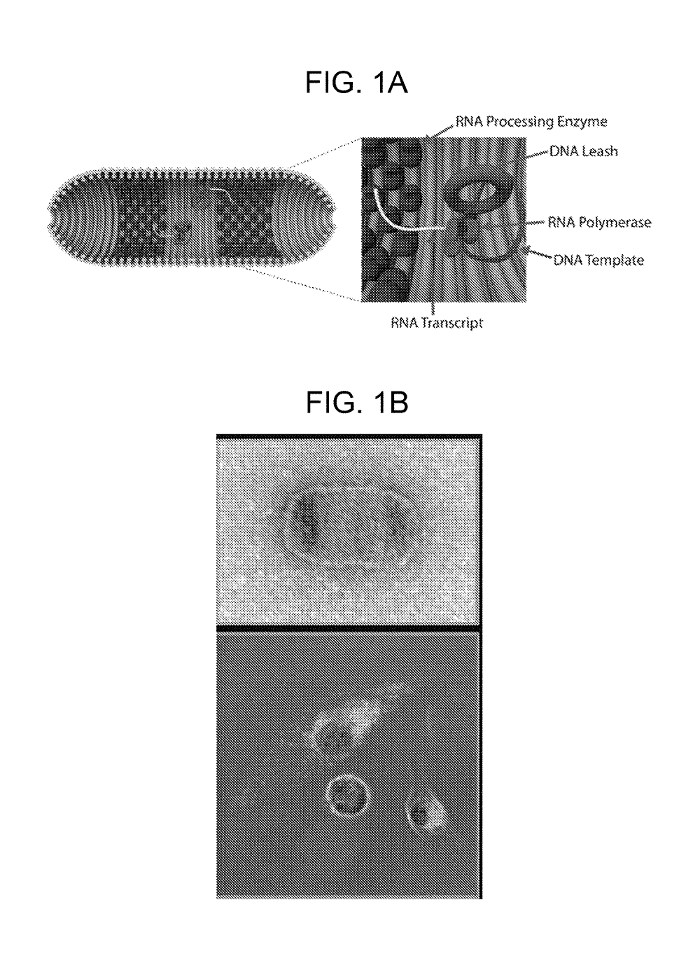

A nucleic acid template (“template”) is a single-stranded or double-stranded nucleic acid comprising a nucleotide sequence that encodes a product (e.g., RNA or protein) of interest. In some embodiments, a template is a DNA template (comprises contiguous deoxyribonucleic acids). In some embodiments, a template is a RNA template (comprises contiguous ribonucleic acids). A template may be linear, having two free termini, or circular, having no free termini. In some embodiments, a template is a circular DNA template, as depicted in FIG. 1A. Circular DNA templates are advantageous for use with a rolling circle replication, amplification or transcription method. Rolling circle methods are processes of unidirectional nucleic acid transcription that can rapidly synthesize multiple copies of circular molecules of DNA or RNA (Gilbert, W. & Dressler, D. Cold Spring Harbor Symp. Quant. Biol., 33: 473-484 (1968); Baker, T. A. & Kornberg, A. DNA Replication (Freeman, N.Y.) (1992), each of which is incorporated by reference herein in its entirety). Rolling circle methods proceed in a linear fashion and use consecutive rounds of replication, amplification or transcription to amplify a target sequence up to about a billion times (see, e.g., Nilsson, M., et al. Science, 265: 2085-88 (1994); Dahl, F. et al., Proc. Nat. Acad. Sci. U.S.A., 101(13): 4548-53 (2004), each of which is incorporated by reference herein in its entirety).

A nucleic acid template, in some embodiments, comprises a promoter (inducible or constitutive) operably linked to a nucleotide sequence encoding a product of interest. A promoter is “operably linked” to a nucleotide sequence if the promoter and the nucleotide sequence are linked in a manner that permits expression of the nucleotide sequence (e.g., in an in vitro transcription/translation system or in an in vivo system). A promoter may be eukaryotic or prokaryotic. In some embodiments, a promoter is a T7 promoter, a T3 promoter or an SP6 promoter. Other promoters are encompassed by the present disclosure.

Nucleic acid templates may also comprises other genetic elements, such as, for example, enhancer sequences, terminators, ribosomal binding sites, etc.

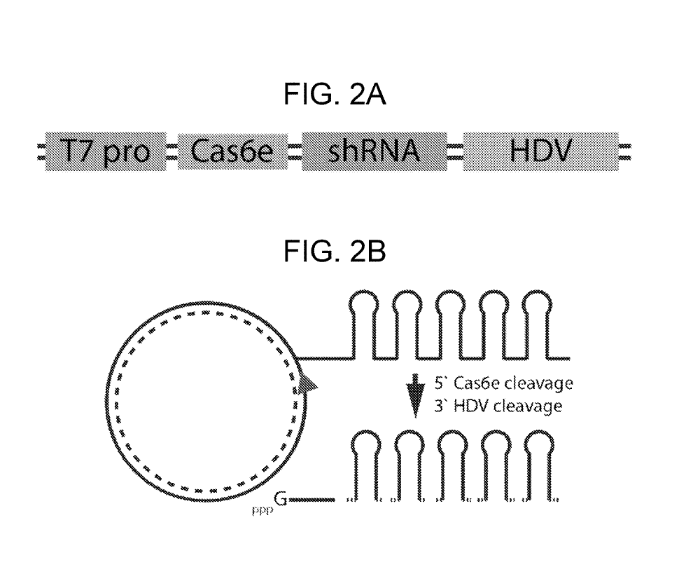

In some embodiments, a nucleic acid template encodes a RNA interference molecule. RNA interference (RNAi) is a post-transcriptional process triggered by the introduction of double-stranded RNA (dsRNA) which leads to gene silencing in a sequence-specific manner. Examples of RNAi molecules include microRNA (miRNA) and small interfering RNA (siRNA) molecules. Short hairpin RNA (shRNA) is another example of a RNAi molecule. FIG. 2A depicts an example of a DNA template comprising a T7 promoter operably linked to a nucleotide sequence encoding an shRNA flanked by endonuclease cleavage sites (Cas6e and HDV). Following transcription (e.g., rolling circle transcription (RCT)) of the DNA template, the polymeric RNA transcript is processed into monomeric form. In some embodiments, processing occurs with an RNA processing molecule. The processing molecule (e.g., an RNA processing molecule), in some embodiments, is linked to an interior surface of the nanostructure.

Thus, in some embodiments, a template includes endonuclease cleavage sites flanking (immediately upstream from and downstream from) a nucleotide sequence encoding a biopolymer of interest. In some embodiments, the nucleotide sequence may be flanked by two ribonuclease recognition sites flanking the 5′ and 3′ ends of the sequence encoding the target. These cleavage sites enable a polymeric RCT RNA transcript or rolling circle replication (RCR) DNA to be processed into monomeric form. Any endonuclease (restriction enzyme) cleavage site and cognate endonuclease may be used, many of which are known in the art.

Examples of endoribonucleases include, without limitation, RNaseH, RNaseIII, Cas6 (e.g., Cas6e from T. Thermophilus) and Cas6 homologs and orthologs of various bacterial species. In some embodiments, a template encodes a ribozyme that mediates self-processing, such as hepatitis delta virus (HDV) (see, e.g., Webb C H, Science 2009, 326(5955):953, incorporated by reference herein). The ribozyme may be a 3′ ribozyme such as HDV or a 5′ ribozyme such as hammerhead ribozyme. Other ribozymes are encompassed by the present disclosure.

Examples of endonucleases include, without limitation, AatII, Acc65I, AccI, AciI, AclI, AcuI, AfeI, AflII, AflIII, AgeI, AgeI-HF™, AhdI, AleI, AluI, AlwI, AlwNI, ApaI, ApaLI, ApeKI, ApoI AscI, AseI, AsiSI, AvaI, AvaII, AvrII, BaeGI, BaeI, BamHI, BamHI-HF™, BanI, BanII, BbsI, BbvCI, BbvI, BccI, BceAI, BcgI, BciVI, BclI, BcoDI, BfaI, BfuAI, BfuCI, BglI, BglII, BlpI, BmgBI, BmrI, BmtI, BpmI, Bpu10I, BpuEI, BsaAI, BsaBI, BsaHI, BsaI, BsaI-HF™, BsalI, BsaWI, BsaXI, BseRI, BseYI, BsgI BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFI , BsrGI, BsrI, BssHII, BssKI, BssSI, BstAPI, BstBI, BstEII, BstNI, BstUI, BstXI, BstYI, BstZ17I, Bsu36I, BtgI, BtgZI, BtsCI, BtsI, BtsIMutI, Cac8I, ClaI, CspCI, CviAII, CviKI-1, CviQI, DdeI, DpnI, DpnII, DraI, DraIII, DraIII-HF™, DrdI , EaeI, EagI, EagI-HF™, EarI, EciI, Eco53kI, EcoNI, EcoO109I, EcoP15I, EcoRI, EcoRI-HF™, EcoRV, EcoRV-HF™, FatI, FauI, Fnu4HI, FokI, FseI, FspEI, FspI, HaeII, HaeIII, HgaI, HhaI, HincII, HindIII, HindIII-HF™, HinfI, HinPlI, HpaI, HpaII, HphI, Hpy166II, Hpy188I, Hpy188III, Hpy99I, HpyAV, HpyCH4III, HpyCH4IV, HpyCH4V, KasI, KpnI, KpnI-HF™, LpnPI, MboI, MboII, MfeI, MfeI-HF™, MluCI, MluI, MlyI, Mmel MnlI, MscI, MseI, MslI, MspAlI, MspI, MspJI, MwoI, NaeI, NalI, NciI, NcoI, NcoI-HF™, NdeI, NgoMIV, NheI, NheI-HF™, NlaIII, NlaIV, NmeAIII, NotI, NotI-HF™, NruI, NsiI, NspI, PacI, PaeR7I, PciI, PflFI, PflMI, PhoI, PleI, PmeI, PmlI, PpuMI, PshAI, PsiI, PspGI, PspOMI, PspXI, PstI, PstI-HF™, PvuI, PvuI-HF™, PvuII, PvuII-HF™, RsaI, RsrII, SacI, SacI-HF™, SacII, SalI, SalI-HF™, SapI, Sau3AI, Sau96I, SbfI, SbfI-HF™, ScaI, ScaI-HF™, ScrFI, SexAI, SfaNI, SfcI, SfiI, SfoI, SgrAI, SmaI, SmlI, SnaBI, SpeI, SphI, SphI-HF™, SspI, SspI-HF™, StuI, StyD4I, StyI, StyI-HF™, SwaI, TaqαI, TfiI, TliI, TseI, Tsp45I, Tsp509I, TspMI, TspRI, Tth111I, XbaI, XcmI, XhoI, XmaI, XmnI, and ZraI. See also EC 3.1.21.3, EC 3.1.21.4 and EC 3.1.21.3.

In some embodiments, a nucleic acid template encodes a messenger RNA (mRNA), which may itself be therapeutic or may encode a therapeutic protein, for example. In some embodiments, the therapeutic RNA is a therapeutic RNA interference (RNAi) molecule. The product of interest, in some embodiments, may be a protein. The protein may also be a prophylactic protein or a diagnostic protein. Examples of therapeutic proteins are known and include, for example, antibodies, enzymes, hormones, inflammatory agents, anti-inflammatory agents, immunomodulatory agents, anti-cancer agents, etc.

Polymerase

A biopolymer synthesis unit comprises a polymerase (an enzyme that synthesizes long chains (polymers) of nucleic acid. The polymerase may be a RNA polymerase or a DNA polymerase. In some embodiments, a biopolymer synthesis unit comprises both a RNA polymerase and a DNA polymerase. RNA synthesis units generally include a RNA polymerase for transcribing an RNA of interest. Examples of RNA polymerases for use herein include, without limitation, T7 RNA polymerase, a T3 RNA polymerase and SP6 RNA polymerase. In some embodiments, RNA polymerase I, II or III may be used, depending on the nucleotide composition of the template and the intended transcribed product. DNA synthesis units generally include an DNA polymerase for replicating a DNA of interest. Examples of DNA polymerases for use herein include, without limitation, DNA polymerase I, II, III, IV or V.

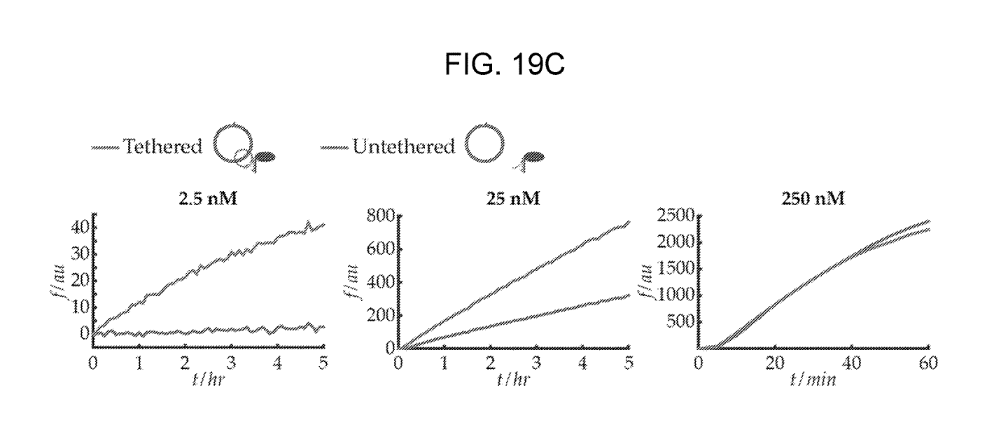

In some embodiments, the polymerase is conjugated to a nucleic acid (e.g., single-stranded nucleic acid) such that the polymerase may be tethered to a leash via an adaptor nucleic acid (or other molecule), as shown for example in FIG. 19A.

Tethers

Molecular components (molecules) of a biopolymer synthesis unit are typically tethered (linked, attached) to an interior surface of a nucleic acid nanocapsule (or other nucleic acid nanostructure). A molecule (e.g., protein or nucleic acid) may be tethered in a site-specific manner to a nucleic acid nanocapsule by any suitable molecular coupling method. In some embodiments, a molecule is tethered to a nanocapsule using a linker, such as a nucleic acid linker or a protein linker.

In some embodiments, a molecule is tethered to a nanocapsule using short single-stranded nucleic acids (e.g., oligonucleotides having a length of shorter than 100 nt) that serve as scaffolds for placement of molecules (see, e.g., Stein et al. Chemphyschem. 12(3), 689-695 (2011); Steinhauer et al. Angew Chem. Int. Ed. Engl. 48(47), 8870-8873 (2009); Stein et al. J. Am. Chem. Soc. 133(12), 4193-4195 (2011); Kuzyk et al. Nature 483(7389), 311-314 (2012); and Ding et al. J. Am. Chem. Soc. 132(10), 3248-3249 (2010); Yan et al. Science 301(5641), 1882-1884 (2003); and Kuzuya et al. Chembiochem. 10(11), 1811-1815 (2009), each of which is incorporated by reference herein). The length of a single-stranded nucleic acid tether may vary. In some embodiments, a nucleic acid tether has a length of 10 to 100 nucleotides. For example, a nucleic acid tether may have a length of 10-90, 10-80, 10-70, 10-60, 10-50, 10-50, 10-40, 10-30, 10-20, 20-100, 20-90, 20-80, 20-70, 20-60, 20-50, 20-40 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides. In some embodiments, a nucleic acid tether is shorter than 100 nucleotides. In some embodiments, a nucleic acid tether is longer than 100 nucleotides.

In some embodiments, a molecule is tethered to a nanocapsule using a nucleic acid (e.g., DNA) aptamer, which adopts a specific secondary structure with high binding affinity for a particular molecular target (see, e.g., Ellington et al. Nature 346(6287), 818-822 (1990); Chhabra et al. J. Am. Chem. Soc. 129(34), 10304-10305 (2007); and Rinker et al. Nat. Nanotechnol. 3(7), 418-422 (2008), each of which is incorporated by reference herein). Nucleic acid aptamers are nucleic acid species that have been engineered through repeated rounds of in vitro selection or equivalently, SELEX (systematic evolution of ligands by exponential enrichment) to bind to various molecular targets such as small molecules, proteins and nucleic acids.

In some embodiments, a molecule is tethered to a nanocapsule using biotin or other accessory molecule. For example, molecules may be tethered to a nanocapsule through chemical biotinylation (see, e.g., Voigt et al. Nat. Nanotechnol. 5(3), 200-203 (2010)). In other embodiments, a molecule may be tethered to a nanocapsule through biotin-streptavidin binding.

In some embodiments, nucleic acids of nanocapsules may be modified (e.g., covalently modified) with a linker (e.g., biotin linker) during assembly of the nanocapsule or via enzymatic means (see, e.g., Jahn et al. Bioconjug. Chem. 22(4), 819-823 (2011), incorporated by reference herein).

In some embodiments, molecules are tethered to a nucleic acid nanocapsule using recombinant genetic engineering. For example, genetically expressed tags, such as a polyhistidine tag, SNAP-tag® (a 20 kDa mutant of the DNA repair protein O6-alkylguanine-DNA alkyltransferase that reacts specifically and rapidly with benzylguanine (B G) derivatives), CLIP-tag™ (a fluorophore conjugated to a cytosine leaving group via a benzyl linker), ACP/MCP-tag (substrates conjugated to the phosphopantetheinyl moiety of Coenzyme A (CoA)), HaloTag® (a modified haloalkane dehalogenase that covalently binds to synthetic ligands comprising a chloroalkane linker) or FLAG® tag (eight amino acids: Asp-Tyr-Lys-Asp-Asp-Asp-Asp-Lys (SEQ ID NO: 1) including an enterokinase-cleavage site) may be used (see, e.g., Sacca et al. Angew Chem. Int. Ed. Engl. 49(49), 9378-9383 (2010), incorporated by reference herein).

In some embodiments, a molecule is tethered to a nanocapsule using chemical groups, such as amines, thiols, azides and/or alkynes. In some embodiments, “click chemistry” reactions are used to tether molecules to a nanocapsule (see, e.g., V. V. Rostovtsev et al. Angew. Chem. Int. Ed., 2002, 41, 2596-2599; F. Himo et al. J. Am. Chem. Soc., 2005, 127, 210-216; and B. C. Boren et al. J. Am. Chem. Soc., 2008, 130, 8923-8930). An example of a click chemistry reaction is the Huisgen 1,3-dipolar cycloaddition of alkynes to azides to form 1,4-disubsituted-1,2,3-triazoles. The copper(I)-catalyzed reaction is mild and very efficient, requiring no protecting groups, and requiring no purification, in many cases. The azide and alkyne functional groups are largely inert towards biological molecules and aqueous environments, which allows the use of the Huisgen 1,3-dipolar cycloaddition in target-guided synthesis and activity-based protein profiling.

In some embodiments, biosynthetic modules include mechanisms that enable actuation of the tethered molecular comments. For example, nucleic acid strand displacement cascades and/or enzymatic reactions are implemented and triggered to actuate molecular components following their localization in a nucleic acid nanocapsule. In some embodiments, these mechanisms are used to control the operation of a nucleic acid nanocapsule (or other nucleic acid nanostructure) in a logical manner, such as sensitizing its operation to environmental inputs.

In some embodiments, a tether is used to link polymerase to a leash (see, e.g., FIG. 19A).

Catenanes

Nucleic acid templates of a biopolymer synthesis unit are typically tethered indirectly to an interior surface of a nucleic acid nanocapsule (or other nucleic acid nanostructure). Indirect tethering may be achieved, for example, by catenating a circular template with another circular molecule that is tethered to a nanocapsule. A catenane is a mechanically-interlocked molecular architecture comprised of two or more interlocked macrocycles (Still, W. C. et al. Tetrahedron 1981, 37, 3981-3996). The connection between two macrocycles of a catenane is referred to as a “mechanical bond.” A unique feature of a mechanical bond is that the bound components do not have to interact with each other: the topological confinement alone is enough to enforce the molecular architecture. Thus, a catenane of a biopolymer synthesis unit serves as a molecular component that allows a nucleic acid template to freely rotate during the transcription process.

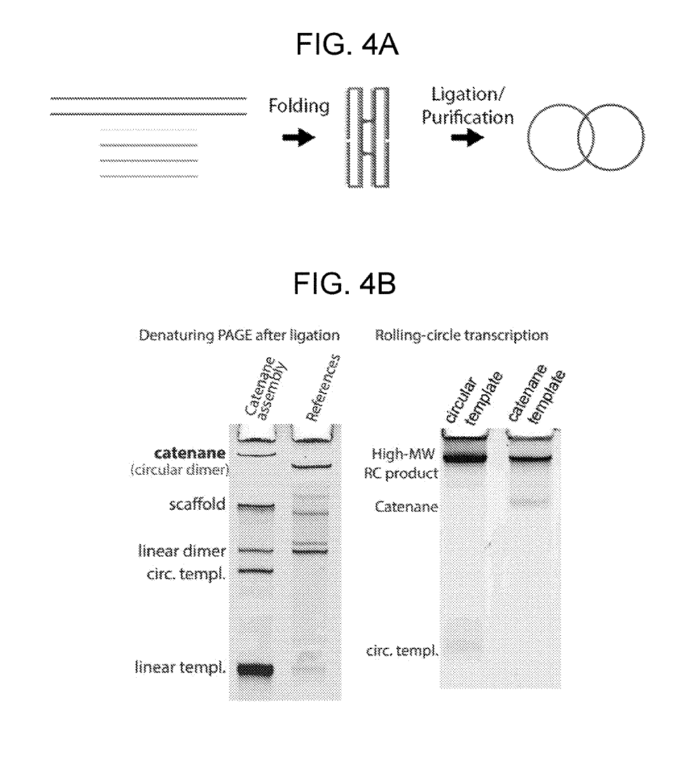

Unlike existing DNA catenanes, which are constructed by circularizing linear DNA strands that are hybridized to each other through complementarity, the DNA catenanes as provided herein are constructed using circular DNA molecules that are sequence-independent relative to one another. Thus, the DNA catenanes of the present disclosure enable free rotation of catenated molecules without having to overcome problems associated with intrinsic binding. An example method for producing a DNA catenane includes formation of DNA nanostructures that enforce the desired topology for two or more linear DNA strands that are circularized and interlocked to one another (see, e.g., FIG. 4A). The sequence independence of the DNA circles is achieved, in this example, by using one or more linear DNA strands that serve as a scaffold and/or braces (shorter single-stranded nucleic acids) so that DNA circles are not required to have sequences complementary to one another. Following ligation, the structure is denatured, and the molecules that formed mechanical bonds can be purified.

The DNA catenanes, as provided herein, include several advantages over the current technology. For example, interlocked DNA circles can be designed not to hybridize and interact with each other so that each DNA circle can freely rotate even when DNA circles are single-stranded; functional moieties and hybridization site can be programmed into DNA circles at specific locations; and multiple DNA catenanes with different topologies can be constructed from the same DNA circles by changing the sequence of DNA braces, for example.

The size of a nucleic acid (e.g., DNA) catenane may vary. The length of a circular nucleic acid may be described in terms of its linearized form. In some embodiments, a circular nucleic acid has a length of 10-5000 nucleotides. For example, a circular nucleic acid may have a length of 10-2500, 10-1000, 10-500, 10-100, 20-5000, 20-2500, 20-1000, 20-500, 20-100, 50-5000, 50-2500, 50-1000, 50-500, 50-100, 100-5000, 100-2500, 100-1000, or 100-500 nucleotides.

Also provided herein are biopolymer synthesis units comprising (or consisting of) a polymerase and a circular nucleic acid template, wherein the polymerase is tethered to (attached to) a circular nucleic acid linker (e.g., a catenane) that is catenated to the circular nucleic acid template. In some embodiments, a biopolymer synthesis unit comprises a major circular nucleic acid to which at least two other minor circular nucleic acids are catenated, wherein at least one of the minor circular nucleic acids is attached to a molecular component, such as a polymerase, or is itself a template, such as a DNA template. Thus, a major circular nucleic acid may be catenated with at least two (e.g., at least 3, 4, 5, 6, 7, 8, 9 or 10) minor circular nucleic acids, each of which may be linked to a molecular component (e.g., polymerase, endonuclease, ribosome, capping enzymes, etc.).

### Additional Molecular Components

Biosynthetic modules, in some embodiments, include other molecules (e.g., in addition to template and polymerase) involved in RNA transcription, post-transcriptional processing and/or and transcript maturation. For example, a biosynthetic module may include nucleoside triphosphates (dNTPs or rNTPs), ligases, ribosomes, endonucleases (DNA endonucleases or RNA endonucleases), endoribonucleases, capping enzymes (an enzyme that catalyzes the attachment of the 5′ cap to mRNA, e.g., RNA triphosphatase, guanylyltransferase (or CE), and/or methyltransferase), multi-protein complexes involved in polyadenylation of mRNA, or any combination thereof.

Biosynthetic modules, in some embodiments, include molecules involved in translation of an RNA transcript. Thus, in some embodiments, a biosynthetic module includes at least one ribosome molecule and/or associated enzymes (e.g., aminoacyl tRNA synthetases, initiation factors, etc.).

In some embodiments, a polymerase is tethered to a leash via an adaptor molecule, such as an adaptor nucleic acid, as shown for example in FIG. 19A. Thus, in some embodiments, a biosynthetic module includes an adaptor (e.g., single-stranded nucleic acid), linking a leash to an a polymerase (e.g., a polymerase conjugated to a separate nucleic acid).

### Nucleic Acid Nanostructures

Biopolymer synthesis units, in some aspects, are localized within a nucleic acid nanocapsule, or other nucleic acid nanostructure. A “nucleic acid nanostructure,” as used herein, is an engineered nanostructure (e.g., having a size of less than 1 μm) assembled from nucleic acids and comprises nucleic acid domains hybridized to each other. Typically, nucleic acid nanostructures are also rationally-designed and artificial (e.g., non-naturally occurring). Nucleic acid nanostructures can self-assemble as a result of sequence complementarity encoded in nucleic acid strands that form that nanostructure. By pairing up complementary segments (through nucleotide base pairing), the nucleic acid strands self-organize under suitable conditions into a predefined nanostructure. Nucleic acid nanostructures may be formed from a plurality (at least two) of nucleic acid strands encoded to hybridize to each other (see, e.g., N. C. Seeman, Nature 421, 427 (2003); International Publication No. WO2013/022694; and International Publication No. WO2014/018675, each of which is incorporated herein by reference), or a nucleic acid nanostructure may be formed from a single strand of nucleic acid (see, e.g., International Application No. PCT/US2016/20893, incorporated herein by reference). Nucleic acid nanostructures typically have dimensionality.

In some embodiments, a nucleic acid nanostructure has a length in each spatial dimension, and is rationally designed to self-assemble (is programmed) into a pre-determined, defined shape that would not otherwise assemble in nature. The use of nucleic acids to build nanostructures is enabled by strict nucleotide base pairing rules (e.g., A binds to T, G binds to C, A does not bind to G or C, T does not bind to G or C), which result in portions of strands with complementary base sequences binding together to form strong, rigid structures. This allows for the rational design of nucleotide base sequences that will selectively assemble (self-assemble) to form nanostructures. For example, a nucleic acid nanostructure may be two dimensional (2D) or three dimensional (3D). A nucleic acid nanocapsule (having a capsule-like structure), as depicted in FIG. 1A, is an example of a 3D nucleic acid nanostructure (nanocapsule).

Nucleic acid structures (e.g., nucleic acid nanostructures) are typically nanometer-scale or micrometer-scale structures (e.g., having a length scale of 1 to 1000 nanometers (nm), or 1 to 10 micrometers (μm)). In some embodiments, a micrometer-scale structure is assembled from more than one nanometer-scale or micrometer-scale structure. In some embodiments, a nucleic acid nanostructure (and, thus, a crystal) has a length scale of 1 to 1000 nm, 1 to 900 nm, 1 to 800 nm, 1 to 700 nm, 1 to 600 nm, 1 to 500 nm, 1 to 400 nm, 1 to 300 nm, 1 to 200 nm, 1 to 100 nm or 1 to 50 nm. In some embodiments, a nucleic acid nanostructure has a length scale of 1, 2, 3, 4, 5, 6, 7, 8, 9 or 10 p.m. In some embodiments, a nucleic acid nanostructure has a length scale of greater than 1000 nm. In some embodiments, a nucleic acid nanostructure has a length scale of 1 μm to 2 μm. In some embodiments, a nucleic acid nanostructure has a length scale of 200 nm to 2 μm, or more.

In some embodiments, a nucleic acid nanostructure (nanocapsule) assembles from a plurality of different nucleic acids (e.g., single-stranded nucleic acids, also referred to as single-stranded tiles, or SSTs (see, e.g., Wei, B. et al. Nature, 485, 623-626, 2012, incorporated herein by reference). For example, a nucleic acid nanostructure may assemble from at least 10, at least 20, at least 30, at least 40, at least 50, at least 60, at least 70, at least 80, at least 90 or at least 100 nucleic acids. In some embodiments, a nucleic acid nanostructure assembles from at least 100, at least 200, at least 300, at least 400, at least 500, or more, nucleic acids. The term “nucleic acid” encompasses “oligonucleotides,” which are short, single-stranded nucleic acids (e.g., DNA) having a length of 10 nucleotides to 200 nucleotides. In some embodiments, an oligonucleotide has a length of 10 to 20 nucleotides, 10 to 30 nucleotides, 10 to 40 nucleotides, 10 to 50 nucleotides, 10 to 60 nucleotides, 10 to 70 nucleotides, 10 to 80 nucleotides, 10 to 90 nucleotides, 10 to 100 nucleotides, 10 to 150 nucleotides, or 10 to 200 nucleotides. In some embodiments, an oligonucleotide has a length of 20 to 50, 20 to 75 or 20 to 100 nucleotides. In some embodiments, an oligonucleotide has a length of 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190 or 200 nucleotides.

In some embodiments, a nucleic acid nanostructure is assembled from single-stranded nucleic acids, double-stranded nucleic acids, or a combination of single-stranded and double-stranded nucleic acids (e.g., includes an end terminal single-stranded overhang).

Nucleic acid nanostructures may assemble, in some embodiments, from a plurality of heterogeneous nucleic acids (e.g., oligonucleotides). “Heterogeneous” nucleic acids may differ from each other with respect to nucleotide sequence. For example, in a heterogeneous plurality that includes nucleic acids A, B and C, the nucleotide sequence of nucleic acid A differs from the nucleotide sequence of nucleic acid B, which differs from the nucleotide sequence of nucleic acid C. Heterogeneous nucleic acids may also differ with respect to length and chemical compositions (e.g., isolated v. synthetic).

The fundamental principle for designing self-assembled nucleic acid structures (e.g., nucleic acid nanostructures) is that sequence complementarity in nucleic acid strands is encoded such that, by pairing up complementary segments, the nucleic acid strands self-organize into a predefined nanostructure under appropriate physical conditions. From this basic principle (see, e.g., Seeman N. C. J. Theor. Biol. 99: 237, 1982, incorporated by reference herein), researchers have created diverse synthetic nucleic acid structures (e.g., nucleic acid nanostructures) (see, e.g., Seeman N. C. Nature 421: 427, 2003; Shih W. M. et al. Curr. Opin. Struct. Biol. 20: 276, 2010, each of which is incorporated by reference herein). Examples of nucleic acid (e.g., DNA) nanostructures, and methods of producing such structures, that may be used in accordance with the present disclosure are known and include, without limitation, lattices (see, e.g., Winfree E. et al. Nature 394: 539, 1998; Yan H. et al. Science 301: 1882, 2003; Yan H. et al. Proc. Natl. Acad. of Sci. USA 100; 8103, 2003; Liu D. et al. J. Am. Chem. Soc. 126: 2324, 2004; Rothemund P. W. K. et al. PLoS Biology 2: 2041, 2004, each of which is incorporated by reference herein), ribbons (see, e.g., Park S. H. et al. Nano Lett. 5: 729, 2005; Yin P. et al. Science 321: 824, 2008, each of which is incorporated by reference herein), tubes (see, e.g., Yan H. Science, 2003; P. Yin, 2008, each of which is incorporated by reference herein), finite two-dimensional and three dimensional objects with defined shapes (see, e.g., Chen J. et al. Nature 350: 631, 1991; Rothemund P. W. K., Nature, 2006; He Y. et al. Nature 452: 198, 2008; Ke Y. et al. Nano. Lett. 9: 2445, 2009; Douglas S. M. et al. Nature 459: 414, 2009; Dietz H. et al. Science 325: 725, 2009; Andersen E. S. et al. Nature 459: 73, 2009; Liedl T. et al. Nature Nanotech. 5: 520, 2010; Han D. et al. Science 332: 342, 2011, each of which is incorporated by reference herein), and macroscopic crystals (see, e.g., Meng J. P. et al. Nature 461: 74, 2009, incorporated by reference herein).

Examples of nucleic acid (e.g., DNA) nanostructures include, but are not limited to, DNA origami structures, in which a long scaffold strand (e.g., at least 500 nucleotides in length) is folded by hundreds (e.g., 100, 200, 200, 400, 500 or more) of short (e.g., less than 200, less than 100 nucleotides in length) auxiliary strands into a complex shape (Rothemund, P. W. K. Nature 440, 297-302 (2006); Douglas, S. M. et al. Nature 459, 414-418 (2009); Andersen, E. S. et al. Nature 459, 73-76 (2009); Dietz, H. et al. Science 325, 725-730 (2009); Han, D. et al. Science 332, 342-346 (2011); Liu, Wet al. Angew. Chem. Int. Ed. 50, 264-267 (2011); Zhao, Z. et al. Nano Lett. 11, 2997-3002 (2011); Woo, S. & Rothemund, P. Nat. Chem. 3, 620-627 (2011); Tørring, T. et al. Chem. Soc. Rev. 40, 5636-5646 (2011). Staple strands are complementary to and bind to two or more noncontiguous regions of a scaffold strand.

In some embodiments, a scaffold strand is 100-10000 nucleotides in length. In some embodiments, a scaffold strand is at least 100, at least 500, at least 1000, at least 2000, at least 3000, at least 4000, at least 5000, at least 6000, at least 7000, at least 8000, at least 9000, or at least 10000 nucleotides in length. The scaffold strand may be naturally occurring or non-naturally occurring. In some embodiments, a single-stranded nucleic acid for assembly of a nucleic acid nanostructure has a length of 500 base pairs to 10 kilobases, or more. In some embodiments, a single-stranded nucleic acid for assembly of a nucleic acid nanostructure has a length of 4 to 5 kilobases, 5 to 6 kilobases, 6 to 7 kilobases, 7 to 8 kilobases, 8 to 9 kilobases, or 9 to 10 kilobases. Staple strands are typically shorter than 100 nucleotides in length; however, they may be longer or shorter depending on the application and depending upon the length of the scaffold strand (a staple strand is typically shorter than the scaffold strand). In some embodiments, a staple strand may be 15 to 100 nucleotides in length. In some embodiments, a staple strand is 25 to 50 nucleotides in length.

In some embodiments, a nucleic acid nanostructure may be assembled in the absence of a scaffold strand (e.g., a scaffold-free structure). For example, a number of oligonucleotides (e.g., less than 200 nucleotides or less than 100 nucleotides in length) may be assembled to form a nucleic acid nanostructure.

In some embodiments, a nucleic acid nanostructure (nanocapsule) is assembled from single-stranded tiles (SSTs) (see, e.g., Wei B. et al. Nature 485: 626, 2012, incorporated by reference herein) or nucleic acid “bricks” (see, e.g., Ke Y. et al. Science 388:1177, 2012; International Publication Number WO 2014/018675 A1, published Jan. 30, 2014, each of which is incorporated by reference herein). For example, single-stranded 2- or 4-domain oligonucleotides self-assemble, through sequence-specific annealing, into two- and/or three-dimensional nanostructures in a predetermined (e.g., predicted) manner. As a result, the position of each oligonucleotide in the nanostructure is known. In this way, a nucleic acid nanostructure may be modified, for example, by adding, removing or replacing oligonucleotides at particular positions. The nanostructure may also be modified, for example, by attachment of moieties, at particular positions. This may be accomplished by using a modified oligonucleotide as a starting material or by modifying a particular oligonucleotide after the nanostructure is formed. Therefore, knowing the position of each of the starting oligonucleotides in the resultant nanostructure provides addressability to the nanostructure.

Other methods for assembling nucleic acid nanostructures are known in the art, any one of which may be used herein. Such methods are described by, for example, Bellot G. et al., Nature Methods, 8: 192-194 (2011); Liedl T. et al, Nature Nanotechnology, 5: 520-524 (2010); Shih W. M. et al, Curr. Opin. Struct. Biol., 20: 276-282 (2010); Ke Y. et al, J. Am. Chem. Soc, 131: 15903-08 (2009); Dietz H. et al, Science, 325: 725-30 (2009); Hogberg B. et al, J. Am. Chem. Soc, 131: 9154-55 (2009); Douglas S. M. et al, Nature, 459: 414-418 (2009); Jungmann R. et al, J. Am. Chem. Soc, 130: 10062-63 (2008); Shih W. M., Nature Materials, 7: 98-100 (2008); and Shih W. M., Nature, 427: 618-21 (2004), each of which is incorporated herein by reference in its entirety.

A nucleic acid nanostructure may be assembled into one of many defined and predetermined shapes including without limitation a capsule, hemi-sphere, a cube, a cuboidal, a tetrahedron, a cylinder, a cone, an octahedron, a prism, a sphere, a pyramid, a dodecahedron, a tube, an irregular shape, and an abstract shape. The nanostructure may have a void volume (e.g., it may be partially or wholly hollow). In some embodiments, the void volume may be at least 25%, at least 30%, at least 35%, at least 40%, at least 45%, at least 50%, at least 55%, at least 60%, at least 65%, at least 70%, at least 75%, at least 80%, at least 85%, at least 90%, at least 91% at least 92%, at least 93%, at least 94%, at least 95%, at least 96%, at least 97%, at least 98%, at least 99%, or more of the volume of the nanostructure.

In some embodiments, nucleic acid nanostructures are rationally designed. A nucleic acid nanostructure is “rationally designed” if the nucleic acids that form the nanostructure are selected based on pre-determined, predictable nucleotide base pairing interactions that direct nucleic acid hybridization (for a review of rational design of DNA nanostructures, see, e.g., Feldkamp U., et al. Angew Chem Int Ed Engl. 2006 Mar 13;45(12):1856-76, incorporated herein by reference). For example, nucleic acid nanostructures may be designed prior to their synthesis, and their size, shape, complexity and modification may be prescribed and controlled using certain select nucleotides (e.g., oligonucleotides) in the synthesis process. The location of each nucleic acid in the structure may be known and provided for before synthesizing a nanostructure of a particular shape. A nanocapsule, rationally designed to resemble the shape of a capsule (e.g., a barrel-shaped central region having a hemisphere dome at each end of the barrel—see, e.g., FIG. 1A), is one example of a particular nucleic acid nanostructure.

Nucleic acid nanostructures of the present disclosure may be two-dimensional or three-dimensional. Two-dimensional nucleic acid structures (e.g., nucleic acid nanostructures) are single-layer planar structures that can be measured along an x-axis and a y-axis. A “layer” of a nucleic acid structure (e.g., nucleic acid nanostructure) refers to a planar arrangement of nucleic acids that is uniform in height. “Height” refers to a measurement of the vertical distance (e.g., along the y-axis) of a structure. “Maximum height” refers to a measurement of the greatest vertical distance of a structure (e.g., distance between the highest point of the structure and the lowest point of the structure). In some embodiments, a nucleic acid layer has a maximum height less than 3 nm (e.g., 1 nm, 1.5 nm, 2 nm, 2.5 nm). A two-dimensional nucleic acid nanostructure is a single-layer structure, thus, in some embodiments, a two-dimensional nucleic acid nanostructure has a planar arrangement of nucleic acids that is uniform in height and has a maximum height less than 3 nm. In some embodiments, a two-dimensional nucleic acid nanostructure has a maximum height of less than 2.5 nm. In some embodiments, a two-dimensional nucleic acid nanostructure has a maximum height of 1 nm to 2.9 nm, or 1 nm to 2.5 nm. In some embodiments, a two-dimensional nucleic acid nanostructure has a maximum height of 1 nm, 1.5 nm, 2 nm or 2.5 nm. Non-limiting examples of two-dimensional nucleic acid structures (e.g., nucleic acid nanostructures) include nucleic acid lattices, tiles and nanoribbons (see, e.g., Rothemund P. W. K., Nature 440: 297, 2006; and Jungmann R. et al., Nanotechnology 22(27): 275301, 2011, each of which is incorporated by reference herein).

Three-dimensional nucleic acid structures (e.g., nucleic acid nanostructures) can be measured along an x-axis, a y-axis and a z-axis. A three-dimensional nucleic acid nanostructure, in some embodiments, has a maximum height equal to or greater than 3 nm. In some embodiments, a three-dimensional nucleic acid nanostructure has a maximum height of greater than 4 nm, greater than 5 nm, greater than 6 nm, greater than 7 nm, greater than 8 nm, greater than 9 nm or greater than 10 nm. In some embodiments, a three-dimensional nucleic acid nanostructure has a maximum height of 3 nm to 50 nm, 3 nm to 100 nm, 3 nm to 250 nm or 3 nm to 500 nm. In some embodiments, a three-dimensional nanostructure may be a multi-layer structure. In some embodiments, a three-dimensional nucleic acid nanostructure comprises 2 to 200, or more, nucleic acid layers. In some embodiments, a three-dimensional nucleic acid nanostructure includes greater than 2, greater than 3, greater than 4, or greater than 5 nucleic acid layers. In some embodiments, a three-dimensional nucleic acid nanostructure comprises 2, 3, 4, 5, 6, 7, 8, 9, 10, 15, 20, 30, 35, 40, 45 or 50 or more nucleic acid layers. A three-dimensional nanostructure may be uniform in height or it may be non-uniform in height. Non-limiting examples of three-dimensional nucleic acid structures (e.g., nucleic acid nanostructures) include nucleic acid capsules and other abstract and/or irregular three-dimensional shapes (see, e.g., Douglas S. M, et al. Nature 459: 414, 2009; Andersen E. D. et al. Nature 459: 73, 2009; Han D. et al. Science 332: 342, 2011; Ke Y. et al., 2011; Wei B., 2012, each of which is incorporated by reference herein). A nanocapsule is an example of a three-dimensional nucleic acid structure.

A three-dimensional nucleic acid nanostructure, in some embodiments, may be assembled from more than one two-dimensional nucleic acid nanostructure (e.g., more than one layer of nucleic acids) or more than one three-dimensional nucleic acid nanostructure (e.g., more than one “pre-assembled” nucleic acid nanostructure that is linked to one or more other “pre-assembled” nucleic acid nanostructure).

“Self-assembly” refers to the ability of nucleic acids (and, in some instances, pre-formed nucleic acid structures (e.g., nucleic acid nanostructures) (nanocapsules)) to anneal to each other, in a sequence-specific manner, in a predicted manner and without external control. In some embodiments, nucleic acid nanostructure self-assembly methods include combining nucleic acids (e.g., single-stranded nucleic acids, or oligonucleotides) in a single vessel and allowing the nucleic acids to anneal to each other, based on sequence complementarity. In some embodiments, this annealing process involves placing the nucleic acids at an elevated temperature and then reducing the temperature gradually in order to favor sequence-specific binding. Various nucleic acid structures (e.g., nucleic acid nanostructures) or self-assembly methods are known and described herein.

Nucleic acid nanostructures, in some embodiments, do not include coding nucleic acid. That is, in some embodiments, nucleic acid nanostructures are “non-coding” nucleic acid nanostructures (the structures are not formed from nucleic acids that encode other molecules). In some embodiments, less than 50% of the nucleic acid sequence in a nucleic acid nanostructure include coding nucleic acid. For example, less than 45%, less than 40%, less than 35%, less than 30%, less than 25%, less than 20%, less than 15%, less than 10% or less than 5% of a nucleic acid nanostructure may include coding nucleic acid sequence.

In some embodiments, a nucleic acid nanostructure comprises or is subsaturated with polyamine polymers, such as polylysine polymers (see, e.g., International Publication No. WO2015/070080). In some embodiments, a nucleic acid nanostructure may comprise or be subsaturated with poly(ethylene imine)-polyethylene glycol (PEI-PEG) copolymers. In some embodiments, a nucleic acid nanostructure may comprise a combination of polyamine polymers and copolymers.

Nucleic acid nanostructures may comprise deoxyribonucleic acid (DNA), ribonucleic acid (RNA), modified DNA, modified RNA, peptide nucleic acid (PNA), locked nucleic acid (LNA), or any combination thereof. In some embodiments, a nucleic acid nanostructure is a DNA nanostructure. In some embodiments, a DNA nanostructure consists of DNA.

A nucleic acid nanocapsule is a nucleic acid nanostructure that forms an exterior surface and an interior compartment (having an interior surface). A nucleic acid (e.g., DNA) nanocapsule may comprise, for example, a (at least one) cylindrical nanostructure having a hemisphere nanostructure (or other cap-like structure) at each end of the cylindrical nanostructure. In some embodiments, a nanocapsule comprises at least two or at least three cylindrical nanostructures linked together to form one large cylinder, each end of the cylinder containing a hemisphere nanostructure (or other cap-like structure). A hemisphere nanostructure may be formed from concentric rings of nucleic acid. An entire capsule, in some embodiments, may be made using a single (or two or three) long scaffold strand and shorter staple strands (e.g., using the DNA origami method).

It should be understood that the shape of a nanocapsule is not intended to be strictly limited to a classic cylindrical shape. The capsule may be cylindrical or circular, for example. Generally, cylindrical capsules have a internal volume that can be calculated by the formula: V=πr²(4/3r+a), where r is the radius of the cylinder and hemispheres and a is the height of the cylindrical part. The surface area formula is SA=2π(2r+a).

The diameter of a nanocapsule may be, for example, 10-500 nm. In some embodiments, a nanocapsule has a diameter of 10, 15, 20, 25, 30, 35, 40, 45, 50, 55, 60, 65, 70, 75, 80, 85. 90, 95 or 100 nm. In some embodiments, a nanocapsule has a diameter of 10-100, 20-100, 30-100, 40-100 or 50-100 nm. In some embodiments, a nanocapsule has a diameter of greater than 500 nm or less than 10 nm.

A nanocapsule may have a spatial resolution of, for example, 10-500 nm. In some embodiments, a nanocapsule has a spatial resolution of 10, 15, 20, 25, 30, 35, 40, 45, 50, 55, 60, 65, 70, 75, 80, 85. 90, 95 or 100 nm. In some embodiments, a nanocapsule has a spatial resolution of 10-100, 20-100, 30-100, 40-100 50-100, 60-100, 70-100, 80-100, or 90-100 nm. In some embodiments, a nanocapsule has a spatial resolution of 10-200, 10-300, 10-400, 10-500, 20-100, 20-200, 20-300, 20-400, 20-500, 30-100, 30-200, 30-300, 30-400, 30-500, 40-100, 40-200, 40-300, 40-400, 40-500, 50-100, 50-200, 50-300, 50-400, 50-500, 75-100, 75-200, 75-300, 75-400, 75-500, 100-200, 100-300, 100-400, 100-500, 200-300, 200-400, 200-500, 300-400, 300-500, or 400-500 nm. In some embodiments, a nanocapsule spatial resolution of greater than 500 nm or less than 10 nm.

A nanocapsule, in some embodiments, comprises a plurality of biopolymer synthesis units. In some embodiments, a nanocapsule comprises 1-1000 biopolymer synthesis units. For example, a nanocapsule may comprise 1-500, 1-100, 1-50, 1-25, 1-10, 2-1000, 2-500, 2-50, 2-25, 2-10, 5-1000, 5-500, 5-100, 5-50, 5-25, 5-10, 10-1000, 10-500, 10-100, 10-50, 10-25, 25-1000, 25-500, 25-100, 25-50, 50-1000, 50-500, 50-100, 100-1000, 100-500 biopolymer synthesis units, each unit containing a co-localized template and polymerase. A nanocapsule, in some embodiments, comprises more than 1000 biopolymer synthesis units.

A product of interest (e.g., a RNA transcript) may be released from a nanocapsule through small holes (pores) in the nanocapsule. Thus, in some embodiments, a nanocapsule comprises a plurality of small (e.g., less than 10 nm) holes through which the product of interest diffuses out.

In some embodiments, a biosynthetic module is not encapsulated in a nucleic acid nanostructure or capsule.

### Methods

The biosynthetic modules of the present disclosure are used, in some embodiments, for the production of biopolymers, such as ribonucleic acid (RNA). These methods comprise, for example, introducing into a cell a biosynthetic module that includes at least one biopolymer synthesizing unit, and incubating the cell under conditions that result in production of RNA. In some embodiments, the biosynthetic module may be introduced into the cytoplasm of a cell. The methods and compositions may be used for a variety of applications, including gene therapy, DNA nanotechnology and immunology.

The cell may be a prokaryotic cell (e.g. bacterial cell, such as Escherichia coli or Staphylococcus aureus) or a eukaryotic cell (e.g., a yeast cell or a mammalian cell). In some embodiments, the cell is a mammalian cell, such as a human cell. In some embodiments, the cell is a tumorigenic cell, such as a cancerous (e.g., metastatic) cell.

Cells may be located in vitro or in vivo, for example, in a subject, such as a human subject. Thus the present disclose provides methods of delivering a product of interest (e.g., a therapeutic, prophylactic or diagnostic ribonucleic acid) to a subject. The methods may comprise, for example, administering to the subject a biosynthetic module (or a plurality of biosynthetic modules), as provided herein. Methods of delivery are known and include intravenous, oral, intranasal, topical, and subdermal delivery methods. Other methods of administration may be used. In some embodiments, a biosynthetic module is injected directly into a cell or a mass of cells (e.g., a tumor).

In some embodiments, the product of interest is a messenger RNA (mRNA) or an RNA interference (RNAi) molecule, such as a shRNA or a siRNA. An mRNA may encode, for example, an antigen (e.g., to induce an immunogenic response) or a therapeutic protein, such as an antibody or antibody fragment. In some embodiments, an mRNA may be a therapeutic molecule.

In some embodiments, the methods comprise incubating a cell (or cells) containing a biosynthetic module (or modules) under conditions that result in production of the RNA of interest. Suitable conditions that result in the production of RNA may be easily determined and depending on factors such as time, temperature, ionic concentration, pH, etc. In vitro condition may be similar to conventional in vitro transcription reaction conditions. In vivo conditions may be physiological conditions.

In some embodiments, the methods comprise introducing a biosynthetic module (or modules) that comprises a RNA polymerase linked to an interior surface of the nanocapsule.

### Additional Embodiments

The present disclosure is also described by the following numbered paragraphs:

1. An engineered nucleic acid nanostructure comprising a biopolymer synthesis unit that comprises a nucleic acid template encoding a product of interest and a polymerase co-localized to an interior surface of the nanostructure.

2. The engineered nucleic acid nanostructure of paragraph 1, wherein the nanostructure is self-assembling.

3. The engineered nucleic acid nanostructure of paragraph 1 or 2, wherein the nanostructure is a deoxyribonucleic acid (DNA) nanostructure.

4. The engineered nucleic acid nanostructure of any one of paragraphs 1-3, wherein the nanostructure is in the shape of a nanocapsule.

5. The engineered nucleic acid nanostructure of any one of paragraphs 1-4, wherein the nanostructure has a spatial resolution of 20-500 nm.

6. The engineered nucleic acid nanostructure of any one of paragraphs 1-5, wherein the polymerase is a ribonucleic acid (RNA) polymerase.

7. The engineered nucleic acid nanostructure of any one of paragraphs 1-6, wherein the polymerase is linked to the interior surface of the nanostructure through chemical coupling using chemical groups.

8. The engineered nucleic acid nanostructure of paragraph 7, wherein the chemical groups are selected from amines, thiols, azides and alkynes.

9. The engineered nucleic acid nanostructure of any one of paragraphs 1-6, wherein the polymerase is linked to the interior surface of the nanostructure through single-stranded nucleic acid linkers.

10. The engineered nucleic acid nanostructure of any one of paragraphs 1-6, wherein the polymerase is linked to the interior surface of the nanostructure through biotin-streptavidin binding.

11. The engineered nucleic acid nanostructure of any one of paragraphs 1-6, wherein the polymerase is linked to the interior surface of the nanostructure through at least one genetically expressed tag.

12. The engineered nucleic acid nanostructure of paragraph 11, wherein the at least one genetically expressed tag is selected from SNAP-tags®, CLIP-tags™, ACP/MCP-tags, HaloTagd® and FLAG® tags.

13. The engineered nucleic acid nanostructure of any one of paragraphs 1-12, wherein the nucleic acid template is a DNA template.

14. The engineered nucleic acid nanostructure of any one of paragraphs 1-13, wherein the nucleic acid template is circular.

15. The engineered nucleic acid nanostructure of any one of paragraphs 1-14, wherein the nucleic acid template is linked to the interior surface of the nanostructure.

16. The engineered nucleic acid nanostructure of paragraph 15, wherein the nucleic acid template is indirectly linked to the interior surface of the nanostructure through catenation with a nucleic acid catenane that is linked to the interior surface of the nanostructure.

17. The engineered nucleic acid nanostructure of paragraph 16, wherein the nucleic acid catenane is a DNA catenane comprising at least two sequence-independent circular DNA molecules.

18. The engineered nucleic acid nanostructure of any one of paragraphs 1-17, wherein the nucleic acid template comprises a promoter operably linked to a nucleotide sequence encoding the product of interest.

19. The engineered nucleic acid nanostructure of paragraph 18, wherein the nucleotide sequence is flanked by ribonuclease recognition sites.

20. The engineered nucleic acid nanostructure of any one of paragraphs 1-19, wherein the product of interest is an RNA.

21. The engineered nucleic acid nanostructure of paragraph 20 wherein the RNA is a messenger RNA (mRNA).

22. The engineered nucleic acid nanostructure of paragraph 20, wherein the RNA is an RNA interference molecule.

23. The engineered nucleic acid nanostructure of paragraph 22, wherein the RNA interference molecule is a short-hairpin RNA (shRNA) molecule or a short-interfering RNA (siRNA) molecule.

24. The engineered nucleic acid nanostructure of any one of paragraphs 20-23, wherein the RNA is a therapeutic RNA.

25. The engineered nucleic acid nanostructure of any one of paragraphs 1-19, wherein the product of interest is a protein.

26. The engineered nucleic acid nanostructure of paragraph 25, wherein the protein is a therapeutic protein, a prophylactic protein or a diagnostic protein.

27. The engineered nucleic acid nanostructure of any one of paragraphs 1-26, wherein the nanostructure further comprises at least one RNA processing molecule.

28. The engineered nucleic acid nanostructure of paragraph 27, wherein the at least one RNA processing molecule is linked to an interior surface of the nanostructure.

29. The engineered nucleic acid nanostructure of paragraph 27 or 28, wherein the at least one RNA processing molecule is selected from DNA endonucleases, RNA endoribonucleases, capping enzymes, ribosomes and ligases.

30. The engineered nucleic acid nanostructure of any one of paragraphs 1-29 comprising at least two, at least five or at least ten RNA-synthesis units.

31. A cell comprising the engineered nucleic acid nanostructure of any one of paragraphs 1-30.

32. The cell of paragraph 31, wherein the nanostructure is located in cytoplasm of the cell.

33. The cell of paragraph 31 or 32, wherein the cell is a mammalian cell.

34. The cell of paragraph 33, wherein the mammalian cell is a human cell.

35. The cell of paragraph 33 or 34, wherein the mammalian cell is a cancerous cell.

36. A method of producing ribonucleic acid (RNA), comprising:

introducing into a cell the engineered nucleic acid nanostructure of any one of paragraphs 1-30; and

incubating the cell under conditions that result in production of RNA. 37. A method of producing ribonucleic acid (RNA), comprising:

introducing into a cell an engineered deoxyribonucleic acid (DNA) nanocapsule comprising a biopolymer synthesis unit that comprises (a) a RNA polymerase linked to an interior surface of the nanocapsule and (b) a circular DNA template comprising a promoter operably linked to a nucleotide sequence encoding a RNA of interest, wherein the circular DNA template is catenated with a nucleic acid catenane that is linked to the interior surface of the nanostructure; and

incubating the cell under conditions that result in production of the RNA of interest. 38. The method of paragraph 37, wherein the nanocapsule further comprises at least one RNA processing molecule linked to an interior surface of the nanostructure.

39. The method of paragraph 38, wherein the at least one RNA processing molecule is selected from DNA endonucleases, RNA endoribonucleases, capping enzymes, ribosomes and ligases.

40. A method of delivering a product of interest to a subject, comprising administering to a subject the nucleic acid nanostructure of any one of paragraphs 1-30, wherein the product of interest is a therapeutic molecule.

41. The method of paragraph 40, wherein the therapeutic molecule is a therapeutic RNA.

42. The method of paragraph 41, wherein the therapeutic RNA is a therapeutic messenger RNA (mRNA).

43. The method of paragraph 42, wherein the therapeutic RNA is a therapeutic RNA interference (RNAi) molecule.

44. A biopolymer synthesis unit comprising a polymerase and a circular nucleic acid template, wherein the polymerase is tethered to a circular nucleic acid linker that is catenated to the circular nucleic acid template.

45. The biopolymer synthesis unit of paragraph 44, wherein the polymerase is conjugated to a single-stranded nucleic acid.

46. The biopolymer synthesis unit of paragraph 44 or 45, wherein an adaptor molecule links the polymerase to the circular nucleic acid linker.

47. The biopolymer synthesis unit of paragraph 46, wherein the adaptor molecule is a single-stranded nucleic acid that is complementary to and binds to both the circular nucleic acid linker and the single-stranded nucleic acid that is conjugated to the polymerase.

48. An engineered nucleic acid nanocapsule comprising the biopolymer synthesis unit of any one of paragraphs 44-47.

49. A cell comprising the engineered nucleic acid nanocapsule of any one of paragraphs 44-48.

50. A method of producing ribonucleic acid (RNA), comprising:

introducing into a cell the engineered nucleic acid nanocapsule of paragraph 48; and

incubating the cell under conditions that result in production of the RNA of interest from the biopolymer synthesis unit.

51. A method of producing ribonucleic acid (RNA), comprising:

introducing into a cell an engineered deoxyribonucleic acid (DNA) nanocapsule comprising a biopolymer synthesis unit that comprises a polymerase and a circular nucleic acid template, wherein the polymerase is tethered to a circular nucleic acid linker that is catenated to the circular nucleic acid template; and

incubating the cell under conditions that result in production of the RNA of interest from the biopolymer synthesis unit.

52. A method of delivering a product of interest to a subject, comprising administering to a subject the engineered nucleic acid nanocapsule of paragraph 48, wherein the product of interest is a therapeutic molecule.

The entire contents of International Publication Number WO 2015/070080 is incorporated by reference herein in its entirety.

### EXAMPLES

### Example 1

In this example, a RNA transcription and maturation method was verified by in vitro transcription of linear short hairpin RNAs (shRNAs) followed by post-transcriptional cleavage at the 5′ and 3′ ends to produce the mature shRNA. Ribonucleic acids (RNAs) have tremendous therapeutic applications, but are notoriously difficult to deliver into target cells in sufficient quantity. Inspired by the poxvirus replication machinery, an RNA extruding nanocapsule was designed in order to actively synthesize therapeutic RNA directly within the cell cytoplasm. The nanocapsule consists of DNA template, RNA polymerase, and endoribonucleases, spatially organized within a DNA origami nanocapsule.

A DNA template was circularized to allow for rolling-circle transcription (RCT) by a RNA polymerase, which increased RNA production yield. Up to 6 copies of RNA polymerase derived from the T7 bacteriophage were anchored in locations adjacent to the DNA template to facilitate their engagement, using SNAP-Tag to attach oligonucleotides to the T7 RNA polymerase. The RNA polymerase transcribes the DNA template in the presence of ribonucleotides to produce RNA concatemers. A DNA catenane was also created as a means to topologically tether the circular DNA template to a DNA-based nanocapsule while still allowing for free rotation of the DNA template during RCT (FIG. 1A). In the sequence of the DNA template, two ribonuclease recognition sites flanking the 5′ and 3′ ends of the RNA target to be produced were encoded (FIG. 2A). This allows RNA multimers produced from RCT to be subsequently cleaved into therapeutically active RNA monomers. The 5′ site is recognized and cleaved by the Cas6e RNA ribonuclease, a 22 kDa endoribonuclease from the Type I CRISPR-Cas bacterial defense system. It catalyzes ssRNA cleavage 3 nt downstream of a 20-29 nt hairpin. Cas6e does not require Mg²⁺ for activity, is relatively active at 37° C. (1=˜2.9 min⁻¹), and supports multiturnover. The endoribonuclease was used to process multimeric RNA transcripts into functional monomers. The 3′ site encodes for a self-cleaving ribozyme from the hepatitis delta virus (HDV). Up to 36 copies of Cas6e at locations flanking the T7 RNAP-template complex were anchored. These ribonucleases recognize and cleave the RNA concatemer transcript, generating the fully matured shRNA sequence. By combining RCT with ribonuclease processing, the RNA production yield was increased and the number of RNA transcripts with 5′ triphosphate caps was minimized (FIG. 2B), which is known to activate inflammatory signaling pathways in cells.

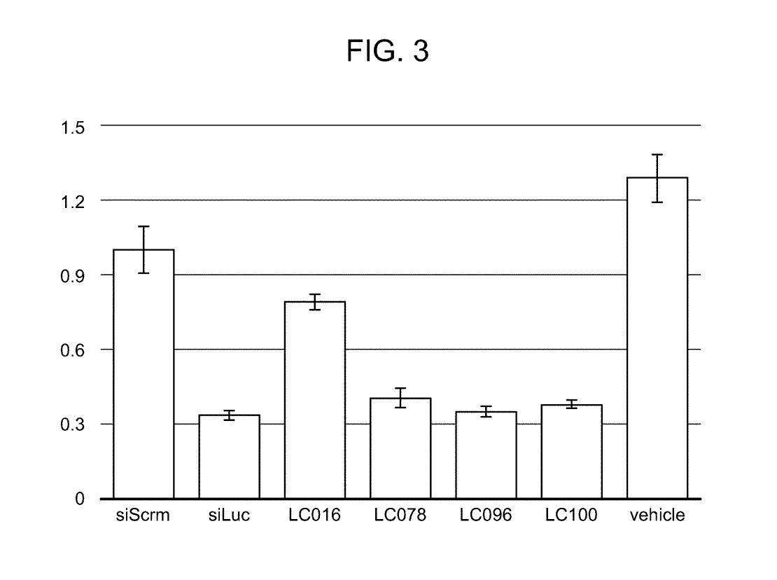

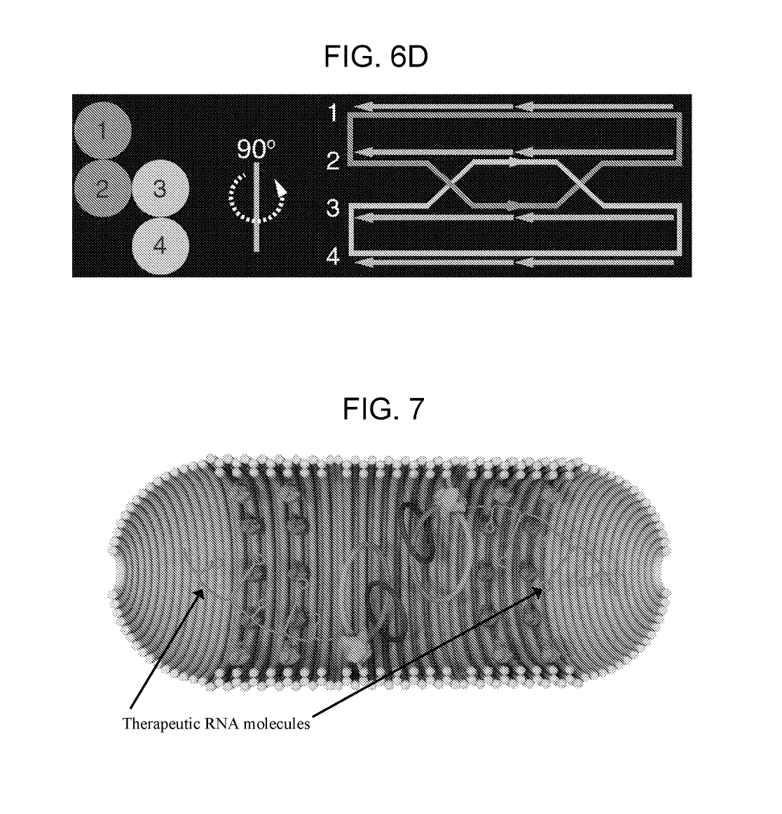

This strategy was applied to screen a range of shRNA sequences targeting a luciferase gene. Functional activity of each in vitro synthesized shRNA sequence was verified by transfection into a luciferase-expressing ovarian cancer cell line (SKOV3-Luc-D3), quantifying the activity of each construct by the reduction in luciferase fluorescence (FIGS. 3, 8A, 8B).

### Example 2

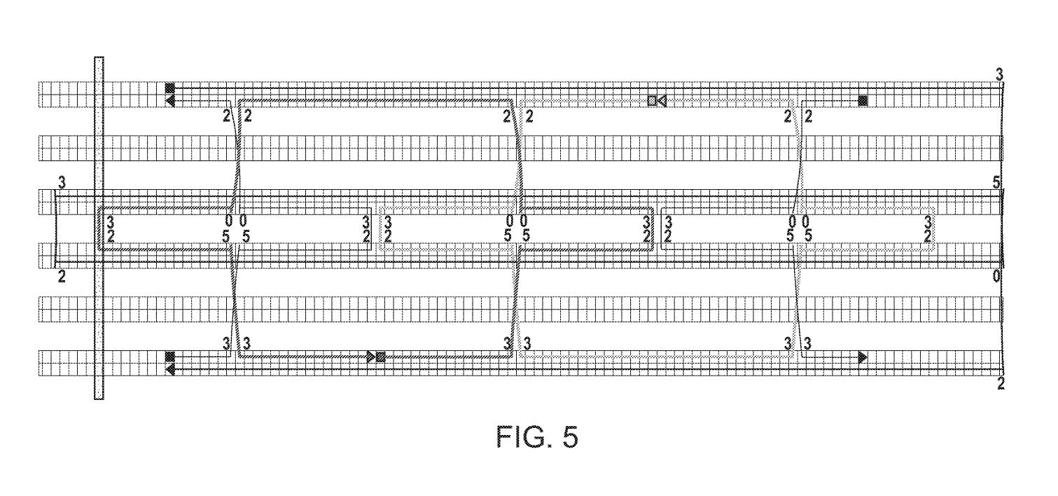

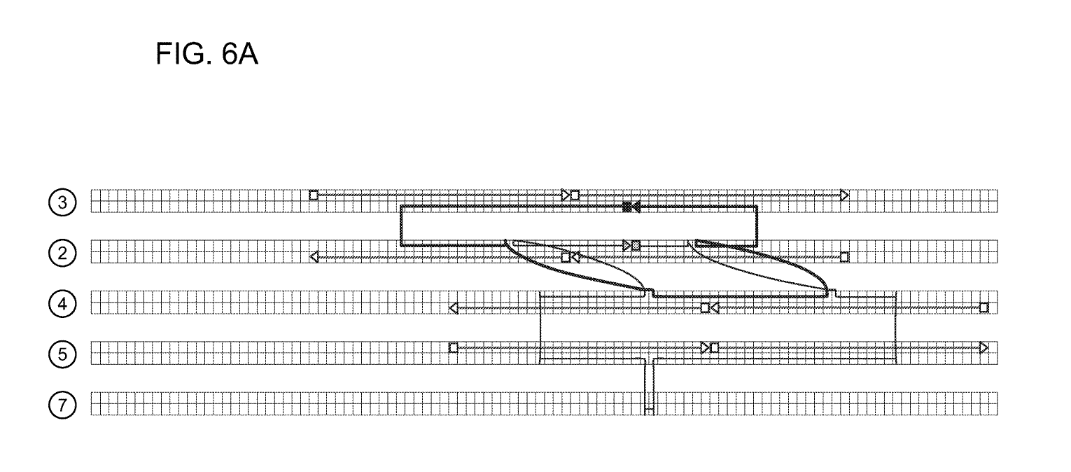

This example describes to example methods of producing a DNA catenane using parallel DNA double crossover motifs as the basic unit to establish crossing between two strands with no complementary sequence. The first example uses a long single-stranded DNA scaffold, similar to the strand used for a DNA origami technique (FIG. 5). The scaffold has complementary sequence to DNA strands to be linked, and the final structure has topology that links circularizing DNA strands in the DNA helical direction. The second example uses short single-stranded DNA braces complementary to the DNA strands to be linked. Unlike the first example, the final structure has topology that links circularizing DNA strands perpendicular to the DNA helical direction (FIGS. 6A-6C).

### Example 3

This example describes an RNA-extruding nanocapsule (nanocapsule) created with DNA origami.

Poxvirus vectors are cytoplasmic expression systems widely used for immunology research and vaccine development. They carry DNA-based RNA polymerase (RNAP) and RNA processing enzymes that make their lifecycle solely in the cytoplasm possible. However, viral vectors have safety concerns associated with their replication, and it is often difficult to engineer them due to their spatial and stoichiometric complexity. With that in mind, a strategy to amplify cytosolic nucleic acid delivery was developed by constructing an artificial system inspired by poxviruses to produce target RNA in the cytoplasm. The resulting RNA-producing nanocapsule was created by tethering multiple copies of RNAP and DNA template inside a DNA origami structure.

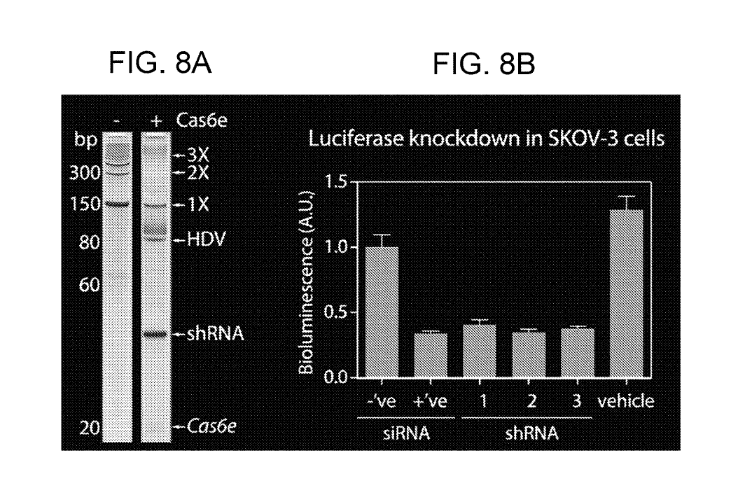

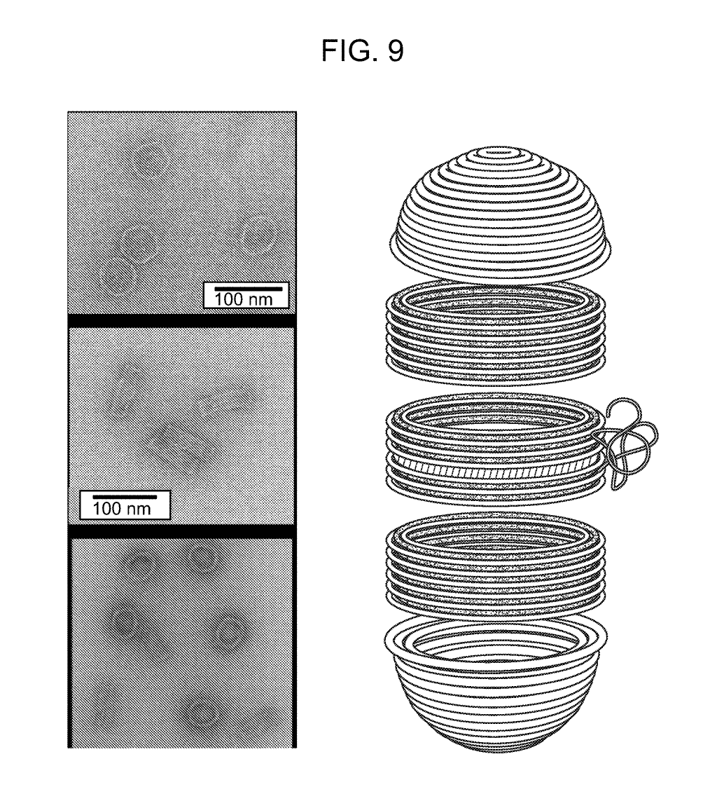

The 3D origami shape selected was the nanocapsule, as it has the ability to carry a large payload, to carry multiple different types of payload (with unique oligo attachments), protect the payload from degradation, and spatial control can be used in targeting ligands. An exemplary nanocapsule is shown in FIG. 9. The structure's ability to carry protein is illustrated in FIG. 10 (top right image). Its integrity is pH-dependent (FIG. 10, bottom).

DNA origami is a unique platform to program complexity. For example, “handles” can be strategically placed within the interior the capsule, where “anti-handles” (for example, imaging labels, RNA polymerase, ribonuclease, and DNA templates) can selectively interact. A schematic overview of the structure is presented in FIG. 1A and an exemplary template sequence is given in FIG. 2A. The target sequence, a shRNA in FIG. 2A, is flanked by two cleavage recognition sites, leading to a 5′ Cas6e cleavage and a 3′ HDV cleavage (FIG. 2B).

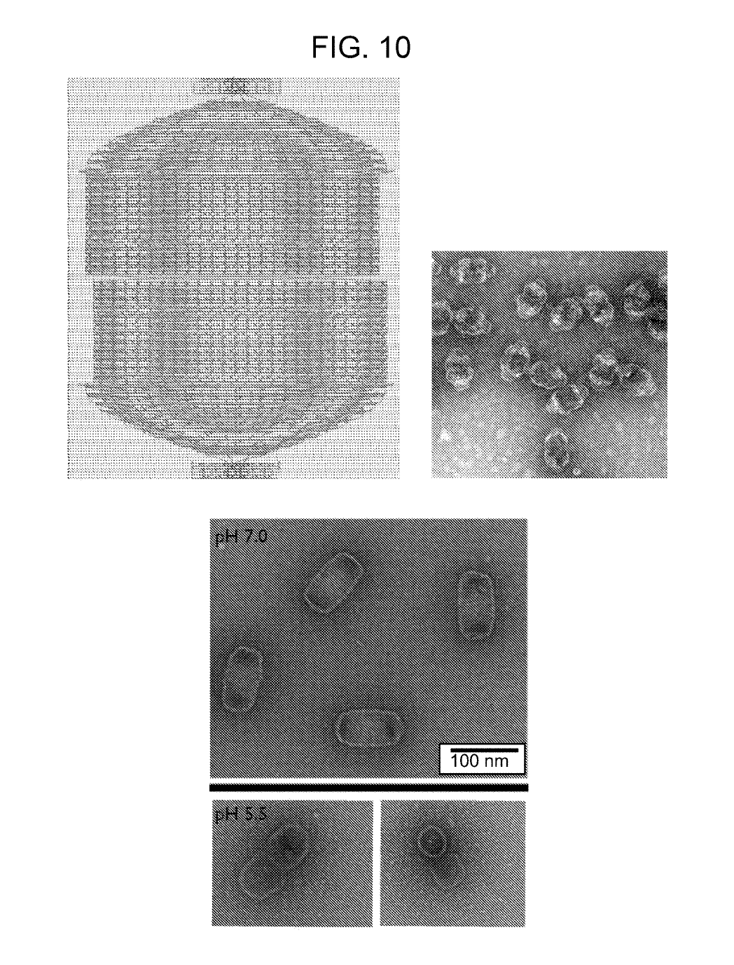

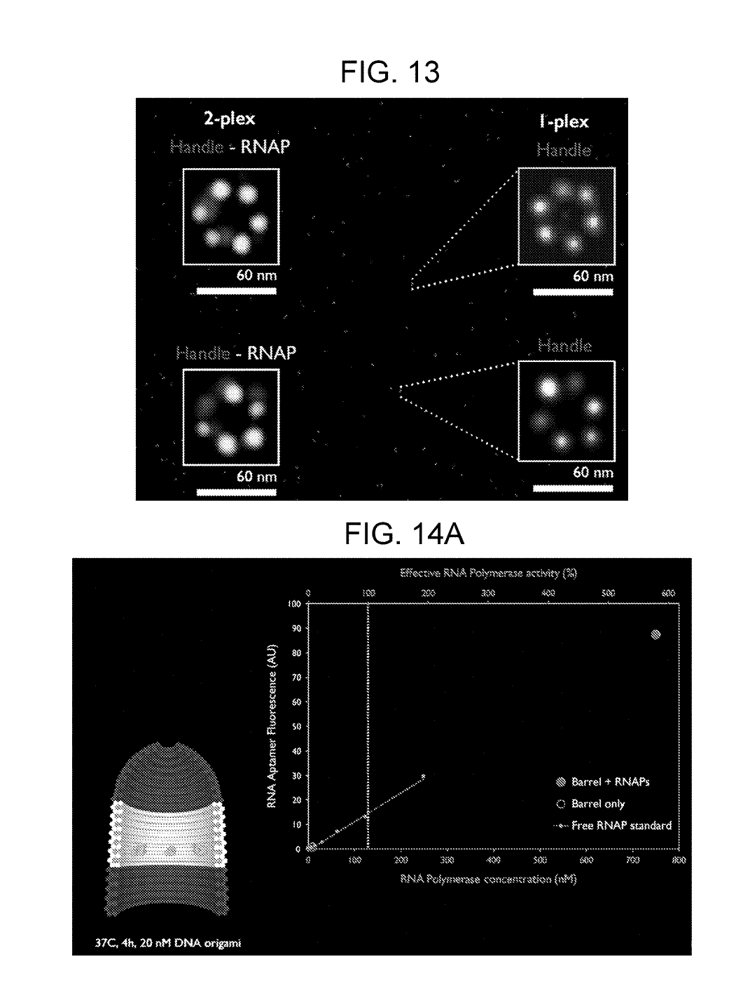

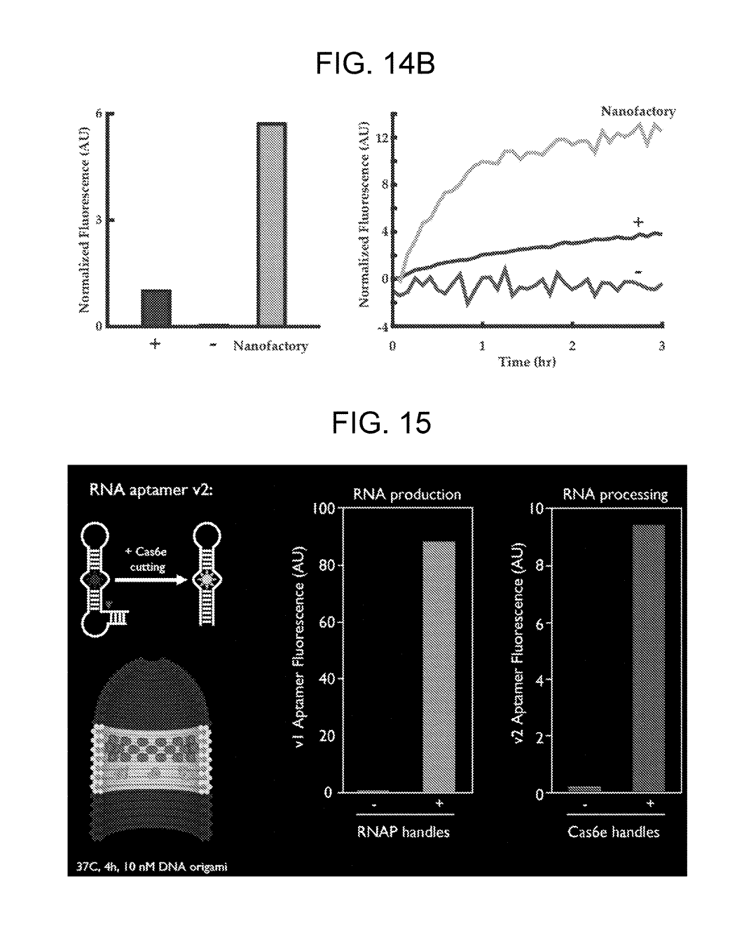

Combining RNA production and processing maximizes the yield of RNA (FIG. 11). The Broccoli aptamer (Filonov et al., J. Am Chem Soc. 136.46:16299-308 (2014)), which binds and activates the fluorescence of DFHBI-1T ((Z)-4-(3,5-difluoro-4-hydroxybenzylidene)-1,2-dimethyl-1H-imidazol-5(4H)-one, was used. RNA production via the nanocapsule system was found to be approximately 6 times more efficient compared to in vitro transcription in solution after 25 minutes (FIG. 14B, left). A sharp transition of RNA production exists for the nanocapsule that is not present for the in vitro transcription in solution (FIG. 14B, right). Further, it was found that DNA barrels contain six binding sites (“handles”) and that RNA polymerase (for example, T7 RNAP) can bind (FIG. 13).

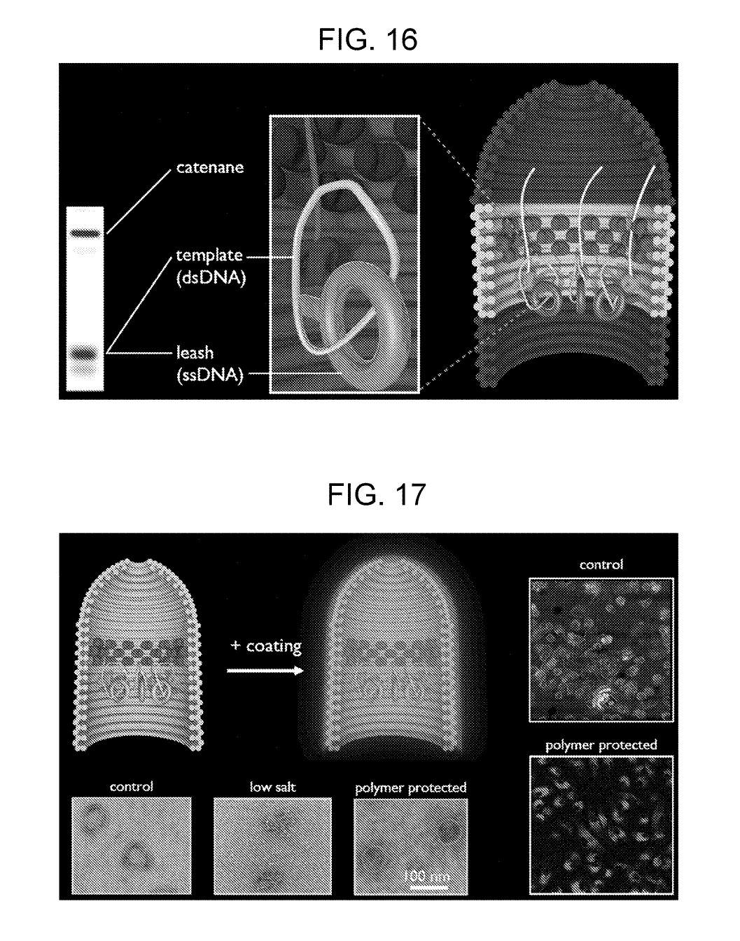

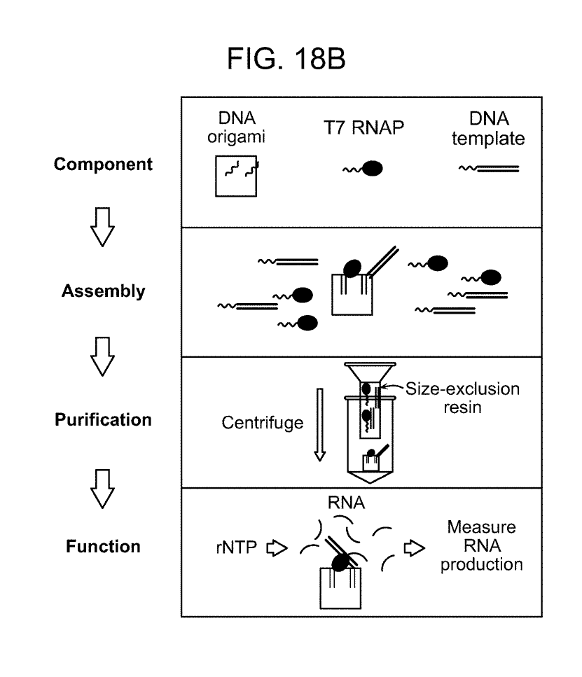

Integrated RNA manufacturing, programmed transcription and processing using handle designs, was shown to yield efficient RNA transcription (FIG. 15). Further, intracellular delivery was improved with the use of polymer coating, which protects against nucleases and low salt conditions, while mediating cell uptake (FIG. 17A).

### Example 4

This example describes an example of a biosynthetic module in the absence of a nanostructure/nanocapsule. As depicted in FIG. 19A, this system comprises a dsDNA template linked to the leash strand via catenane formation. The adaptor strand hybridizes to both the leash strand and the oligo-conjugated T7 RNA polymerase (RNAP) to form a biosynthetic module. Formation was confirmed by SDS-PAGE (FIG. 19B).

Using the broccoli aptamer, the fluorescence signal from different concentrations of tethered (oligo-conjugated T7 RNAP, adaptor, and circular dsDNA template with a leash strand linked via catenane formation) or untethered (oligo-conjugated T7 RNAP, adaptor, and circular dsDNA template without a leash strand) systems was measured. The results are shown in FIG. 19C. At high concentrations (250 nM), the observed fluorescence signal was comparable between the two systems. Tethered transcription performed better than untethered transcription as the concentration decreased. At low concentrations (2.5 nM), there was no fluorescence signal observed for untethered transcription, while the tethered transcription produced observable fluorescence signal.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An engineered nucleic acid nanocapsule comprising a biopolymer synthesis unit that comprises a nucleic acid template encoding a product of interest and a polymerase co-localized to an interior surface of the nanocapsule.

2. The engineered nucleic acid nanocapsule of claim 1, wherein the nanostructure is self-assembling.

3. The engineered nucleic acid nanocapsule of claim 1, wherein the nanostructure is a deoxyribonucleic acid (DNA) nanostructure.

4. The engineered nucleic acid nanocapsule of claim 1, wherein the nanostructure is cylindrical.

5. The engineered nucleic acid nanocapsule of claim 1, wherein the nanostructure has a spatial resolution of 20-500 nm.

6. The engineered nucleic acid nanocapsule of claim 1, wherein the polymerase is a ribonucleic acid (RNA) polymerase.

7. The engineered nucleic acid nanocapsule of claim 1, wherein the polymerase is linked to the interior surface of the nanostructure through chemical coupling using chemical groups.

8. The engineered nucleic acid nanocapsule of claim 7, wherein the chemical groups are selected from amines, thiols, azides and alkynes.

9. The engineered nucleic acid nanocapsule of claim 1, wherein the polymerase is linked to the interior surface of the nanostructure through single-stranded nucleic acid linkers.

10. The engineered nucleic acid nanocapsule of claim 1, wherein the polymerase is linked to the interior surface of the nanostructure through biotin-streptavidin binding.

11. The engineered nucleic acid nanocapsule of claim 1, wherein the polymerase is linked to the interior surface of the nanostructure through at least one genetically expressed tag.

12. The engineered nucleic acid nanocapsule of claim 11, wherein the at least one genetically expressed tag is selected from SNAP-tags®, CLIP-tags™, ACP/MCP-tags, HaloTagd® and FLAG® tags.

13. The engineered nucleic acid nanocapsule of claim 1, wherein the nucleic acid template is a DNA template.

14. The engineered nucleic acid nanocapsule of claim 1, wherein the nucleic acid template is circular.

15. The engineered nucleic acid nanocapsule of claim 1, wherein the nucleic acid template is linked to the interior surface of the nanocapsule.

16. The engineered nucleic acid nanocapsule of claim 15, wherein the nucleic acid template is indirectly linked to the interior surface of the nanocapsule through catenation with a nucleic acid catenane that is linked to the interior surface of the nanocapsule.

17. The engineered nucleic acid nanocapsule of claim 16, wherein the nucleic acid catenane is a DNA catenane comprising at least two sequence-independent circular DNA molecules.

18. The engineered nucleic acid nanocapsule of claim 1, wherein the nucleic acid template comprises a promoter operably linked to a nucleotide sequence encoding the product of interest.

19. The engineered nucleic acid nanocapsule of claim 18, wherein the nucleotide sequence is flanked by ribonuclease recognition sites.

20. The engineered nucleic acid nanocapsule of claim 1, wherein the product of interest is an RNA.

21. The engineered nucleic acid nanocapsule of claim 20 wherein the RNA is a messenger RNA (mRNA).

22. The engineered nucleic acid nanocapsule of claim 20, wherein the RNA is an RNA interference molecule.

23. The engineered nucleic acid nanocapsule of claim 22, wherein the RNA interference molecule is a short-hairpin RNA (shRNA) molecule or a short-interfering RNA (siRNA) molecule.

24. The engineered nucleic acid nanocapsule of claim 20, wherein the RNA is a therapeutic RNA.

25. The engineered nucleic acid nanocapsule of claim 1, wherein the product of interest is a protein.

26. The engineered nucleic acid nanocapsule of claim 25, wherein the protein is a therapeutic protein, a prophylactic protein or a diagnostic protein.

27. The engineered nucleic acid nanocapsule of claim 1, wherein the biopolymer synthesis unit further comprises at least one RNA processing molecule.

28. The engineered nucleic acid nanocapsule of claim 27, wherein the at least one RNA processing molecule is linked to an interior surface of the nanocapsule.

29. The engineered nucleic acid nanostructure of claim 27, wherein the at least one RNA processing molecule is selected from DNA endonucleases, RNA endoribonucleases, capping enzymes, ribosomes and ligases.

30. The engineered nucleic acid nanocapsule of claim 1 comprising at least two, at least five or at least ten biopolymer synthesis units.

31. A cell comprising the engineered nucleic acid nanocapsule of claim 1.

32. The cell of claim 31, wherein the nanocapsule is located in cytoplasm of the cell.

33. The cell of claim 31, wherein the cell is a mammalian cell.

34. The cell of claim 33, wherein the mammalian cell is a human cell.

35. The cell of claim 33, wherein the mammalian cell is a cancerous cell.

36. A method of producing ribonucleic acid (RNA), comprising:
introducing into a cell the engineered nucleic acid nanocapsule of claim 1; and
incubating the cell under conditions that result in production of RNA from the biopolymer synthesis unit.

37. A method of producing ribonucleic acid (RNA), comprising:
introducing into a cell an engineered deoxyribonucleic acid (DNA) nanocapsule comprising a biopolymer synthesis unit that comprises (a) a RNA polymerase linked to an interior surface of the nanocapsule and (b) a circular DNA template comprising a promoter operably linked to a nucleotide sequence encoding a RNA of interest, wherein the circular DNA template is catenated with a nucleic acid catenane that is linked to the interior surface of the nanostructure; and
incubating the cell under conditions that result in production of the RNA of interest from the biopolymer synthesis unit.

38. The method of claim 37, wherein the biopolymer synthesis unit further comprises at least one RNA processing molecule linked to an interior surface of the nanostructure.

39. The method of claim 38, wherein the at least one RNA processing molecule is selected from DNA endonucleases, RNA endoribonucleases, capping enzymes, ribosomes and ligases.

40. A method of delivering a product of interest to a subject, comprising administering to a subject the nucleic acid nanostructure of claim 1, wherein the product of interest is a therapeutic molecule.

41. The method of claim 40, wherein the therapeutic molecule is a therapeutic RNA.

42. The method of claim 41, wherein the therapeutic RNA is a therapeutic messenger RNA (mRNA).

43. The method of claim 42, wherein the therapeutic RNA is a therapeutic RNA interference (RNAi) molecule.

44. A biopolymer synthesis unit comprising a polymerase and a circular nucleic acid template, wherein the polymerase is tethered to a circular nucleic acid linker that is catenated to the circular nucleic acid template.

45. The biopolymer synthesis unit of claim 44, wherein the polymerase is conjugated to a single-stranded nucleic acid.

46. The biopolymer synthesis unit of claim 45, wherein an adaptor molecule links the polymerase to the circular nucleic acid linker.

47. The biopolymer synthesis unit of claim 46, wherein the adaptor molecule is a single-stranded nucleic acid that is complementary to and binds to both the circular nucleic acid linker and the single-stranded nucleic acid that is conjugated to the polymerase.

48. An engineered nucleic acid nanocapsule comprising the biopolymer synthesis unit of claim 44.

49. A cell comprising the engineered nucleic acid nanocapsule of claim 48.

50. A method of producing ribonucleic acid (RNA), comprising:
introducing into a cell the engineered nucleic acid nanocapsule of claim 48; and
incubating the cell under conditions that result in production of the RNA of interest from the biopolymer synthesis unit.

51. A method of producing ribonucleic acid (RNA), comprising:
introducing into a cell an engineered deoxyribonucleic acid (DNA) nanocapsule comprising a biopolymer synthesis unit that comprises a polymerase and a circular nucleic acid template, wherein the polymerase is tethered to a circular nucleic acid linker that is catenated to the circular nucleic acid template; and
incubating the cell under conditions that result in production of the RNA of interest from the biopolymer synthesis unit.

52. A method of delivering a product of interest to a subject, comprising administering to a subject the engineered nucleic acid nanocapsule of claim 48, wherein the product of interest is a therapeutic molecule.
